(19) United States
(12) Patent Application Publication
(10) Pub. No.: US 2015/0057500 A1
(43) Pub. Date: Feb. 26, 2015

(54) System for Connecting and Disconnecting A Main Connector and A Main Control Unit of An Endoscope

(71) Applicant: EndoChoice Inc., Alpharetta, GA (US)

(72) Inventors: Golan Salman, Atlit (IL); Igal Shteiman, Kfar Yona (IL); Amram Aizenfeld, Ramot Menashe (IL); Stephan Wieth, Klein Nordende (DE)

(21) Appl. No.: 14/468,189

(22) Filed: Aug. 25, 2014

Related U.S. Application Data
(60) Provisional application No. 61/870,144, filed on Aug. 26, 2013.
(60) Provisional application No. 61/968,436, filed on Mar. 21, 2014.

(51) Int. Cl. A61B 1/00

(52) U.S. Cl. CPC A61B 1/00124; A61B 1/00006
USPC 600/132

(57) ABSTRACT

The present specification describes systems for connecting/disconnecting a main connector of an endoscope assembly to the main control unit. In an embodiment, the system includes an elongated protruding member located on the main connector which is adapted to fit into a female fitting member placed in an opening on the surface of the main control unit wherein said female fitting member is coupled to a motor, which on activation causes the female fitting member to rotate and engage with the elongated protruding member. In an embodiment, the system includes a male bayonet component located on the main connector for connecting with a female bayonet component placed in an opening on a surface of the main control unit, wherein said female fitting member is coupled to a slidable switch through a crankshaft in the main control unit.

### CROSS-REFERENCE TO RELATED APPLICATIONS

The present application relies on, for priority, U.S. Provisional Patent Application No. 61/870,144, entitled “System for Connecting and Disconnecting a Connector To An Endoscope Main Control Unit”, and filed on Aug. 26, 2013, and U.S. Provisional Patent Application No. 61/968,436, entitled “System for Connecting and Disconnecting a Main Connector and A Main Control Unit of An Endoscope”, filed on Mar. 21, 2014, both of which are herein incorporated by reference in their entirety.

### FIELD

The present specification generally relates to systems for connecting a main connector to a main control unit of an endoscope and disconnecting/releasing the same.

### BACKGROUND

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy and others. Endoscopes may be inserted into the body's natural orifices or through an incision in the skin.

An endoscope is usually an elongated tubular shaft, rigid or flexible, having a video camera or a fiber optic lens assembly at its distal end. The shaft is connected to a handle, which sometimes includes an ocular for direct viewing. Viewing is also possible via an external display. Various surgical tools may be inserted through a working channel in the endoscope for performing different surgical procedures.

In an electronic endoscope system, the main control unit, which is used to process data from an endoscope and the endoscope itself are generally set up for use as discrete units, where the endoscope is a device that can be detachably attached to the main control unit. The handle section of endoscope is usually coupled to a main control unit with the help of an umbilical tube and a main connector. The main control unit houses the controls required for enabling various functionalities in the endoscope and for displaying the images of internal organs captured by the endoscope on a display screen contained in the main control unit or on external monitors. The exchange of all signals and other inputs between the endoscope assembly and main control unit is performed through the connection between the main connector of the endoscope assembly and the main control unit. Therefore the connection between the main connector and main control unit is very critical and should be securely managed. Additionally, any communication should be initiated only once a secure connection between the endoscope assembly and the main control unit has been established.

U.S. patent application Ser. No. 11/746,138 discloses “a connecting mechanism for an electronic endoscope system that is used for detachably connecting a scope and a processor unit of said electronic endoscope system, said connecting mechanism comprising: a rotational cylinder that locks a scope-side connector section to a processor-side connector section by turning said rotational cylinder, when the scope-side connector section is docked with said processor-side connector section; a linear movable member that is moved linearly along a tangent of the arc of movement of said rotational cylinder in cooperation with the rotation of said rotational cylinder; and a sensor that detects whether said linear movable member has arrived at a predetermined position; wherein said predetermined position corresponds to a locking position where said scope-side connector section and said processor-side connector section are locked together by said rotational cylinder.”

There is a need in the art for a system which allows quick and easy connection of the main connector to a main control unit. There is also a need for a system which can enable release/disconnection of the main connector from the main control unit by applying minimal physical force that is also convenient to use.

### SUMMARY

The following embodiments and aspects thereof are described and illustrated in conjunction with systems, tools and methods, which are meant to be exemplary and illustrative, not limiting in scope. The present specification is directed towards systems for connecting a main connector to a main control unit of an endoscope and disconnecting/releasing the same.

The present specification is directed towards a connection system for connecting a connector to a main control unit of an endoscope, said connector comprising: an elongated protruding member adapted to fit into a receiving structure positioned on a surface of said main control unit, wherein said receiving structure is coupled to a motor which, when activated, causes said receiving structure to rotate and engage or disengage with said elongated protruding member.

In an embodiment, the elongated protruding member has an outer periphery and comprises at least one protruding pin on said outer periphery, wherein said at least one protruding pin is adapted to fit into the grooves of said receiving structure when said receiving structure is rotated.

Optionally, the elongated protruding member is a locking guide of the main connector.

In an embodiment, the receiving structure is placed in an opening of the main control unit and the opening is for receiving the locking guide of main connector.

In an embodiment, the motor is coupled to the receiving structure through a motor holding section and a shaft. Optionally, the motor is coupled to a rear portion of said receiving structure.

Optionally, the connection system further comprises a sensor device which detects a presence of said elongated protruding member and activates the motor. In an embodiment, the sensor device is positioned on a front section of the said elongated protruding member such that, when the elongated protruding member is inserted into the receiving structure, the sensor device is activated. In an embodiment, the sensor device is positioned on an outer portion of the receiving structure.

Optionally, the receiving structure is a bayonet connector.

In an embodiment, the connection system further comprises a switch adapted to activate the motor for disengaging a connection between said receiving structure and said elongated protruding member.

In another embodiment, the present specification is directed toward an endoscope comprising: an insertion tube having a lens assembly at its distal end; a control handle connected to said insertion tube and comprising the means to control various functionalities in endoscope; an umbilical tube that connects the control handle to a main connector comprising an elongated protruding member; a main control unit having a female fitting member which is configured to receive said elongated protruding member of the main connector and is coupled to a motor which when activated, rotates said female fitting member to engage/disengage with said elongated protruding member.

In an embodiment, the elongated protruding member comprises at least one protruding pin on its outer periphery which is adapted to fit into the grooves of said female fitting member when said female fitting member is rotated.

Optionally, the elongated protruding member is the locking guide of the main connector.

In an embodiment, the female fitting member is placed in an opening of the main control unit and said opening is for receiving the locking guide of main connector. In an embodiment, the motor is coupled to the female fitting member through a motor holding section and a shaft. In an embodiment, the motor is coupled to the rear portion of said female fitting member.

Optionally, the system further comprises a sensor device which detects the presence of said elongated protruding member and activates the motor which causes the female fitting member to rotate and engage with the elongated protruding member. In an embodiment, the sensor device is positioned on the front section of the shaft that couples with the female fitting member such that when the elongated protruding member is inserted into the female fitting member the sensor device is activated. In an embodiment, the sensor device is positioned on the outer tip of the female fitting member.

Optionally, the female fitting member is a bayonet connector.

Optionally, a switch is used to activate the motor for disengaging tire connection.

In another embodiment, the present specification is directed toward a system for disconnecting a connector portion of an endoscope from a control unit, comprising: an elongated protruding member coupled to the connector portion; and a control unit having an opening adapted to receive the elongated protruding member coupled to the connector portion, wherein said opening comprises a shaft coupled to a motor, which when activated, is adapted to cause said shaft to move longitudinally and eject said elongated protruding member, thereby disengaging the endoscope from said main control unit. In an embodiment, the shaft comprises a threaded portion.

Optionally, the elongated protruding member is a locking guide of the connector portion.

Optionally, the opening is a locking opening of the control unit. Optionally, the shaft is D-shaped and the opening in the control unit is D-shaped.

In an embodiment, the motor is activated through an operation button, positioned on the control unit.

In yet another embodiment, the present specification is directed towards an endoscope comprising: an insertion tube having a video camera or a lens assembly at its distal end; a control handle connected to said insertion tube and comprising the means to control various functionalities in endoscope; an umbilical tube that connects the control handle to a main connector comprising an elongated protruding member; and, a main control unit comprising an opening adapted to receive said elongated protruding member for establishing a connection with the main connector; wherein said opening comprises a shaft coupled to a motor, which when activated, causes said shaft to move longitudinally and push out said elongated protruding member to disengage the connection.

Optionally, the shaft comprises a threaded portion.

Optionally, the elongated protruding member is the locking guide of the main connector. Optionally, the locking opening is a locking opening of the main control unit.

Optionally, the shaft is D-shaped and the corresponding opening in the main control unit to accommodate said shaft is also D-shaped.

Optionally, the motor is activated through an operation button, positioned on the control unit, to disengage the connection.

In yet another embodiment, the present specification is directed toward a system for connecting a main connector to a main control unit of an endoscope, said connector comprising a locking guide which is adapted to fit into a female bayonet connector placed in the locking guide opening on a surface of said main control unit, wherein said female bayonet connector is coupled to a motor which when activated, causes said female bayonet connector to rotate and engage/disengage with at least one protruding pin positioned on said locking guide.

In an embodiment, the motor is coupled to the female bayonet connecter through a motor holding section and a shaft. In an embodiment, the motor is coupled to the rear portion of the female bayonet connector.

Optionally, the system further comprises a sensor device which detects the presence of said locking guide and activates the motor which causes the female bayonet connector to rotate and engage with the locking guide. Still optionally, the sensor device is positioned on the front section of the shaft that couples with the female bayonet connector such that when the locking guide is inserted into the female fitting member the sensor device is activated. Still optionally, the sensor device is positioned on the outer tip of the female fitting member.
Optionally, a switch which is used to activate the motor for disengaging the connection.

In still yet another embodiment, the present specification is directed toward an endoscope having a connection portion for connecting to a control unit, the connector portion comprising a male fitting member which is adapted to fit into a female fitting member placed in an opening on a surface of the control unit, wherein said female fitting member is coupled to a slidable switch through a crankshaft located in the control unit.

Optionally, the female fitting member and the slidable switch are located on a main connector housing on the main control unit.

Optionally, the male fitting member is a bayonet connector. In an embodiment, the male fitting member comprises an electrical connection portion.

Still optionally, the female fitting member is a bayonet connector. In an embodiment, the female fitting member is placed in an opening of the main control unit, and the opening is for receiving a utility cable.

In an embodiment, the slidable switch is moved linearly in a vertical or horizontal direction.

Optionally, the crankshaft is a forged structure fixedly attached to the female fitting member and/or the slidable switch. Still optionally, the crankshaft is fixedly attached to said female fitting member and/or said slidable switch using screws.

In an embodiment, the endoscope further comprises a slider groove over which the slidable switch can slide.

Optionally, the endoscope is activated by moving the slidable switch in a linear direction from a first position to a second position after the connection portion is placed in the control unit.

Optionally, the endoscope is activated by moving the slidable switch in a linear direction from an unlocked position to a locked position after the connection portion is placed in the control unit.

In yet another embodiment, the present specification is directed toward an endoscope comprising: an insertion tube having a video camera or a lens assembly at its distal end; a control handle connected to said insertion tube and comprising the means to control various functionalities in endoscope; an umbilical tube that connects the control handle to a main connector having a mate fitting member; and, a main control unit having a female fitting member which is configured to receive said male fitting member positioned on the main connector and is coupled to a slidable switch through a crankshaft.

In an embodiment, the female fitting member and the slidable switch are located in the main connector housing of main control unit.

Optionally, the male filling member is a bayonet connector. Still optionally, the make fitting member comprises an electrical connector port ion of the main connector.

Optionally, the female fitting member is a bayonet connector. Still optionally, the female fitting member is placed in an opening of the main control unit, and wherein said opening is for receiving a utility cable.

In an embodiment, the slidable switch is moved linearly in a vertical or horizontal direction.

Optionally, the crankshaft is a forged structure permanently fixed to the female fitting member and/or the slidable switch. Still optionally, the crankshaft is attached to said female fitting member and/or said slidable switch using screws.

In an embodiment, the endoscope further comprises a slider groove over which the slidable switch can slide.

In an embodiment, the endoscope is activated by moving the slidable switch in a linear direction from a first position to a second position after the main connector is placed in the main control unit. In another embodiment, the endoscope is activated by moving the slidable switch in a linear direction from an unlocked position to a locked position after the main connector is placed in the main control unit.

In yet another embodiment, the present specification is directed toward a system for connecting a proximal connection portion of an endoscope to a control unit of an endoscope, the connection portion comprising a male fitting member which is adapted to fit into a female fitting member placed in an opening on a surface of the main control unit, wherein said female fitting member is coupled to a crankshaft that is adapted to be operated by activating an electrical motor.

In an embodiment, a movement of said crankshaft causes the female fitting member to rotate and lock or unlock with said male fitting member.

Optionally, the electrical motor is manually operated through a switch provided on the control unit.

Optionally, the system comprises a sensor device which detects that the male fitting member is connected to the female fitting member and, based on said detection, activates said electrical motor to lock the male fitting member to the female fitting member.

Optionally, said crankshaft and said electrical motor are located in the control unit.

Optionally, the male fitting member is a bayonet connector. Still optionally, the male fitting member comprises an electrical connection portion.

Optionally, the female fitting member is a bayonet connector. Still optionally, the female fitting member is placed in an opening of the control unit, wherein said opening is adapted to receive a utility cable.

Optionally, the crankshaft is a forged structure permanently fixed to the female fitting member and/or the slidable switch. Still optionally, the crankshaft is attached to said female fitting member and/or said slidable switch using screws.

In still yet another embodiment, the present specification is directed toward an endoscope comprising: an insertion tube having a video camera or a lens assembly at its distal end; a control handle connected to said insertion tube and comprising the means to control various functionalities in endoscope; an umbilical tube that connects the control handle to a main connector having a male fitting member; and, a main control unit having a female fitting member placed in an opening on a surface of the main control unit, wherein said female fitting member is configured to receive said male fitting member positioned on the main connector and is coupled to a crankshaft which is operated by activating an electrical motor.

In an embodiment, the movement of said crankshaft causes the female fitting member to rotate and lock/unlock with said male fitting member.

Optionally, the electrical motor is operated through an operations switch provided on the main control unit.

Optionally, the system comprises a sensor device which detects that the male fitting member is docked with the female fitting member and accordingly activates said electrical motor to lock them.

Optionally, said crankshaft and said electrical motor are located in the main control unit.

Optionally, the male fitting member is a bayonet connector. Still optionally, the male fitting member comprises an electrical connection portion.

Optionally, the female fitting member is a bayonet connector. Still optionally, the female fitting member is placed in an opening of the control unit, wherein said opening is adapted to receive a utility cable.

Optionally, the crankshaft is a forged structure permanently fixed to the female fitting member and/or the slidable switch. Still optionally, the crankshaft is attached to said female fitting member and/or said slidable switch using screws.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1A illustrates a system for connecting a main connector to a main control unit of an endoscope with the help of an elongated protruding member coupled to the main connector in accordance with an embodiment of the present specification;

FIG. 1B illustrates a main connector securely connected to a main control unit, in accordance with an embodiment of the present specification;

FIG. 2 illustrates a main connector housing on the main control unit front panel, in accordance with an embodiment of the present specification;

FIG. 3A illustrates a bayonet connector, in accordance with an embodiment of the present specification;

FIG. 3B illustrates another view of the bayonet connector, shown in FIG. 3B, in accordance with an embodiment of the present specification;

FIG. 4 illustrates a bayonet shaft, in accordance with an embodiment of the present specification;

FIG. 5 illustrates a locking guide being inserted into a female bayonet connector, in accordance with an embodiment of the present specification;

FIG. 6 illustrates a rear view of a main control unit panel when a main connector is connected to a main connector housing, in accordance with an embodiment of the present specification;

FIG. 7A illustrates a female bayonet connector positioned over a locking opening in a main connector housing and a main connector comprising a locking guide, in accordance with an embodiment of the present specification;

FIG. 7B illustrates a locking guide placed at the entrance of a female bayonet connector housed within a locking opening in a main connector housing, in accordance with an embodiment of the present specification;

FIG. 7C illustrates a locking guide locked within a female bayonet connector mounted on a locking opening in a main connector housing, in accordance with an embodiment of the present specification;

FIG. 8A illustrates a main connector being released from a main control unit, in accordance with an embodiment of the present specification;

FIG. 8B illustrates another view of a main connector being released from a main control unit, in accordance with an embodiment of the present specification;

FIG. 9A illustrates a cross-sectional view of a main connector connected to a main control unit, in accordance with an embodiment of the present specification;

FIG. 9B illustrates a cross-sectional view of another embodiment of the present specification showing the main connector connected to a main control unit;

FIG. 9C illustrates a cross-sectional view of a main connector being released from a main control unit, in accordance with an embodiment of the present specification;

FIG. 10 illustrates a system for connecting a main connector with a main control unit by using a bayonet connection mechanism and a crankshaft arrangement in accordance with another embodiment of the present specification;

FIG. 11 illustrates the main control unit without the outer panel of main connector housing, with a slidable switch in a first position in accordance with an embodiment of the present specification;

FIG. 12 illustrates a two-dimensional front view of the main control unit without the outer panel of the main connector housing, connected to the main connector, and with a slidable switch in a first position in accordance with an embodiment of the present specification;

FIG. 13 illustrates the main control unit without the outer panel of main connector housing and with a slidable switch in a second position, in accordance with an embodiment of the present specification;

FIG. 14 illustrates an embodiment of the main control unit without the outer panel of the main connector housing and with the main connector connected to the main control unit, with the slidable switch in a second position;

FIG. 15A illustrates the movement of a female bayonet connector and a crankshaft caused by a change in the position of the slidable switch in accordance with an embodiment of the present specification;

FIG. 15B illustrates the movement of a female bayonet connector and a crankshaft caused by a change in the position of the slidable switch in accordance with an embodiment of the present specification;

FIG. 15C illustrates the movement of a female bayonet connector and a crankshaft caused by a change in the position of the slidable switch in accordance with an embodiment of the present specification;

FIG. 15D illustrates the movement of a female bayonet connector and a crankshaft caused by a change in the position of the slidable switch in accordance with an embodiment of the present specification;

FIG. 15E illustrates the movement of a female bayonet connector and a crankshaft caused by a change in the position of the slidable switch in accordance with an embodiment of the present specification;

FIG. 16A illustrates a main connector and the female bayonet component separately in an embodiment of the present specification;

FIG. 16B illustrates a side view of the main connector and the female bayonet component separately in accordance with an embodiment of the present specification;

FIG. 16C illustrates a side view of the main connector wherein the male bayonet component is engaged with a female bayonet component in accordance with an embodiment of the present specification;

FIG. 16D illustrates an enlarged view of the female bayonet component present on the main control unit and the male bayonet component present on the main connector in accordance with an embodiment of the present specification;

FIG. 17 illustrates an embodiment of the system disclosed in present specification, with the main connector housing covered with an outer panel and with the main connector of the scope partially inserted into the main control unit, where the slidable switch is in a first position; and,

FIG. 18 illustrates an embodiment of the system disclosed in present specification, with the main connector housing covered with an outer panel and with the main connector of the scope fully inserted into the main control unit, where the slidable switch is in a second position.

### DETAILED DESCRIPTION

The present specification discloses a system for connecting a main connector of an endoscope to a main control unit which enables various functionalities in an endoscope. The system also enables easy release of the main connector from the main control unit with the application of minimal manual force. The present specification discloses various systems that use bayonet connection mechanisms to connect the main connector with the main control unit of endoscope.

In an embodiment, the system includes a main control unit comprising a receiving structure which is adapted to receive an elongated protruding member coupled to a main connector of endoscope. In an embodiment, the receiving structure is a female bayonet connector mounted over the surface of a main control unit which is adapted to receive an elongated protruding member such as a locking guide of the main connector, such that the locking guide locks up with the female bayonet connector with the help of a protruding pin present on its outer periphery, which is locked into the grooves of female bayonet connector.

In an embodiment, the female bayonet connector is coupled with a bayonet motor at its back end, which, on activation, rotates the female bayonet connector around the protruding pin present on the outer periphery of the locking guide to lock the protruding pin into its grooves. In an embodiment, the bayonet motor is coupled to the female bayonet connector through a motor holding section and a shaft which assist in firmly securing the entire bayonet system within the main control unit and in transforming the motor activity into circular motion of the female bayonet connector.

One of ordinary skill in the art would appreciate that there may be multiple methods to activate the bayonet motor described in the above embodiment. In an embodiment, a sensor device is deployed such that when the locking guide docks with the female bayonet connector, the sensor device communicates this information to the bayonet motor and activates the same to lock the main connector into the main control unit. In an embodiment, the sensor device is positioned on the outer periphery of the female bayonet connector. In an alternate embodiment, the sensor device is present on a shaft that couples the bayonet motor to the female bayonet connector such that when the locking guide is inserted into the female bayonet connector, the sensor device is pressed which, in turn, activates the bayonet motor to lock the main connector with the main control unit of endoscope.

In the above embodiment, the female bayonet connector can be mounted at various positions on the main control unit. In an embodiment, the female bayonet connector is mounted onto an opening of a main connector housing on the main control unit which is configured to house the main connector of the endoscope assembly.

In another embodiment of the present specification, the system includes a main control unit with a female bayonet connector mounted over its surface adapted to receive and releasably fit into a locking configuration with a male bayonet connector present on the main connector of the scope. In the above embodiment, the female bayonet connector comprises a cylindrical section which is coupled to a slidable switch via a crankshaft system. In an embodiment, the slidable switch is located on the surface of the main control unit and is in a first position when the endoscope is not in use. In the first position, in an embodiment, the slidable switch is open and the corresponding cylindrical section of the female bayonet connector is in an unlocked position. To connect the endoscope assembly with a main control unit, the main connector of endoscope is first placed in the main connector housing or socket located on the front panel of the main control unit. A male bayonet connector located on the main connector of the endoscope docks with the female bayonet connector located on the surface of main connector housing on the main control unit. Subsequently, the slidable switch is moved in a vertical direction to a second position. In an embodiment, moving the slidable switch to a second position causes the cylindrical section of female bayonet connector to rotate and connect with the male bayonet component of the main connector.

The crankshaft couples the slidable switch with the female bayonet connector and translates the linear motion of slidable switch into the circular motion of the female bayonet component. The presence of crankshaft system reduces the physical effort required to rotate the female bayonet connector as compared to a lock lever based bayonet connection mechanism, which requires effort and manual rotation. The presence of a slidable switch in the main connector housing provides a convenient connection mechanism and is thus very convenient for the user to operate the endoscope. In an embodiment, a sliding groove is also provided in the main connector housing through which the slidable switch can be moved in a vertical direction.

One of ordinary skill in the art can appreciate that in the embodiment described above, while the slidable switch is configured to move in a vertical direction it can also be configured to move in a horizontal direction. In other embodiments, a push button may also be used to activate the crankshaft system. One would also appreciate that while the above embodiment discloses a slidable switch which is positioned in the main connector housing, the switch can, in other embodiments, be positioned at some location outside of the main connector housing.

In an embodiment, the male bayonet connector is located on the electric connector portion of the main connector of an endoscopic system and the female bayonet connector is positioned over the surface of the utility cable opening in the main control unit. However, one of ordinary skill in the art can appreciate that the male and female bayonet components can be located at multiple positions within the main connector and main control unit, respectively. The position of the slidable switch and the accompanying crankshaft system can be adjusted accordingly.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

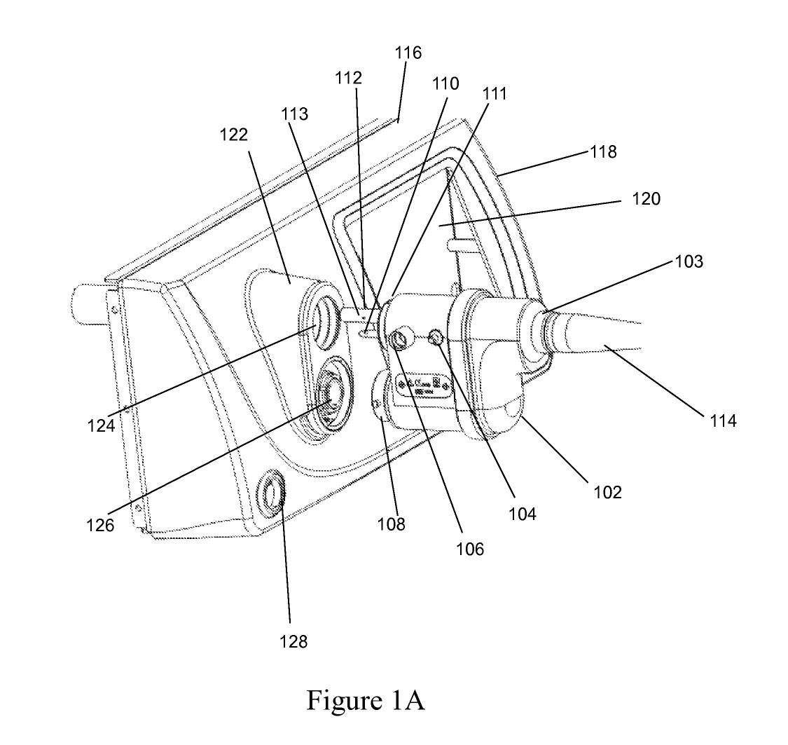

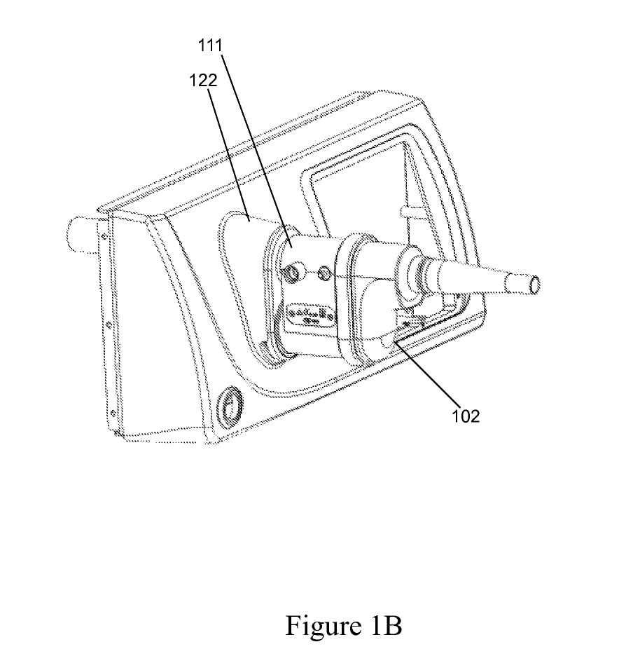

FIG. 1A illustrates a system for connecting a main connector to a main control unit of an endoscope with the help of an elongated protruding member coupled to the main connector in accordance with an embodiment of the present specification. As illustrated, in an embodiment, the main connector 102 may comprise a jet connector 104, wherein the jet connector 104 is typically connected to a fluid supplier to provide fluid to a jet opening in an endoscope tip, a water bottle connector 106, wherein the water bottle connector 106 is typically engaged to a water supplier, such as a water bottle or hospital facilities, to provide fluid to an insufflation and/or irrigation system placed within the endoscope tip, an electric connector 108, wherein the electric connector 108 connects between electronics components within the endoscope, such as but not limited to, sensors, illuminators, handle of the endoscope and the main control unit to provide electricity to the various components, a gas channel 110, wherein the gas channel 110 typically provides gas flow to the tip of the endoscope. In an embodiment, the main connector 102 comprises an elongated protruding member 112 which is adapted to fit into a receiving structure present on the surface of main control unit. In an embodiment, the elongated protruding member 112 is a locking guide of the endoscope, wherein the locking guide 112 includes at least one pin 113, placed on a periphery of the locking guide 112.

In conventional endoscopes, a locking guide and/or integrated light guide 112 is a fiber optic tube/pin which is used to direct the light from an external light source to a distal tip of endoscope. Hence, in an embodiment, the light guide 112 serves the dual purpose of directing the light from an external source to distal tip of endoscope and also acts as a means for connecting the main connector to main control unit. In an embodiment where the endoscope comprises video cameras instead of a fiber optic based lens assembly, the light guide pin is redundant as it is not used to direct the light from external source. In this case, in the above embodiment, it is used only as a means for connecting the main connector to the main control unit as described in above embodiment.

In an embodiment, a distal end 103 of the main connector 102 is connected to a utility cable 114. In an embodiment, a proximal end 111 of the main connector is connected to the main control unit.

In an embodiment, the main control unit 116 governs power transmission to the endoscope's tip section, such as for the tip section's viewing elements and illuminators. The main control unit 116 further controls one or more fluid, liquid and/or suction pumps which supply corresponding functionalities to the endoscope. In an embodiment, one or more input devices, such as a keyboard, a touch screen, at least one monitor and the like are connected to the main control unit 116 for the purpose of human interaction with the main control unit 116. In an embodiment, an input device, such as a keyboard or a touch screen is integrated with the main control unit 116 in the same casing.

In an embodiment, the main control unit 116 comprises a front panel 118 having a screen 120 for displaying operation information regarding an endoscopy procedure when the endoscope is in use. In an embodiment, the screen 120 is configured to display images and/or video streams received from the viewing elements of a multiple viewing element endoscope. In an embodiment, the screen 120 is also configured to display a user interface for allowing a human operator to set various features of the endoscopy system.

In an embodiment, optionally, the video streams received from the different viewing elements of a multi-viewing element endoscope can be displayed separately on at least one monitor (not seen) uploading information from the main control unit 116, either side-by-side or interchangeably (namely, the operator may switch between views from the different viewing elements manually). In an embodiment, these video streams are processed by the main control unit 116 to combine them into a single, panoramic video frame, based on an overlap between fields of view of the viewing elements. In an embodiment, two or more displays are connected to the main control unit 116, each for displaying a video stream from a different viewing element of the multi-viewing element endoscope. Video processing by the main control unit 116 is described in U.S. patent application No. 263,896, entitled “Video Processing in a Compact Multi-Viewing Element Endoscope System”, and filed on Apr. 28, 2014, which is herein incorporated by reference in its entirety.

In an embodiment, the main control unit 116 comprises a main connector housing 122 for receiving the main connector 102. In an embodiment, the main connector housing 122 comprises a first section 124 for connecting with locking guide 112 and gas channel 110 and a second section 126 for receiving the electric connector portion 108.

In an embodiment, the main connector housing 122 comprises a receiving structure mounted in the locking opening of the first section 124 which is adapted to receive the elongated protruding member/locking guide 112 coupled to the main connector and lock the protruding pin 113 present on its outer periphery in its grooves such that the main connector is firmly connected with the main control unit. In an embodiment, the said receiving structure is a female fitting member such as a female bayonet connector. The front panel 118 further comprises a button 128 for switching the main control unit 116 on or off.

FIG. 1B illustrates a main connector securely connected to a main control unit, in accordance with an embodiment of the present specification. As shown in FIG. 1B, a proximal end 111 of the main connector 102 is connected to the main control unit 116 and the elongated protruding member/locking guide (shown as 112 in FIG. 1A) is lockably inserted into a receiving structure/female bayonet connector placed in a locking opening of the main connector housing 122. In an embodiment, the female bayonet connector is coupled with a bayonet motor at its back end, which, on activation, rotates the female bayonet connector around the protruding pin present on the outer periphery of the locking guide to lock the pin into its grooves. In an embodiment, the bayonet motor is coupled to the female bayonet connector through a motor holding section and a shaft which, in an embodiment, assist in firmly securing the entire bayonet system within the main control unit and in transforming the motor activity into circular motion of the female bayonet connector.

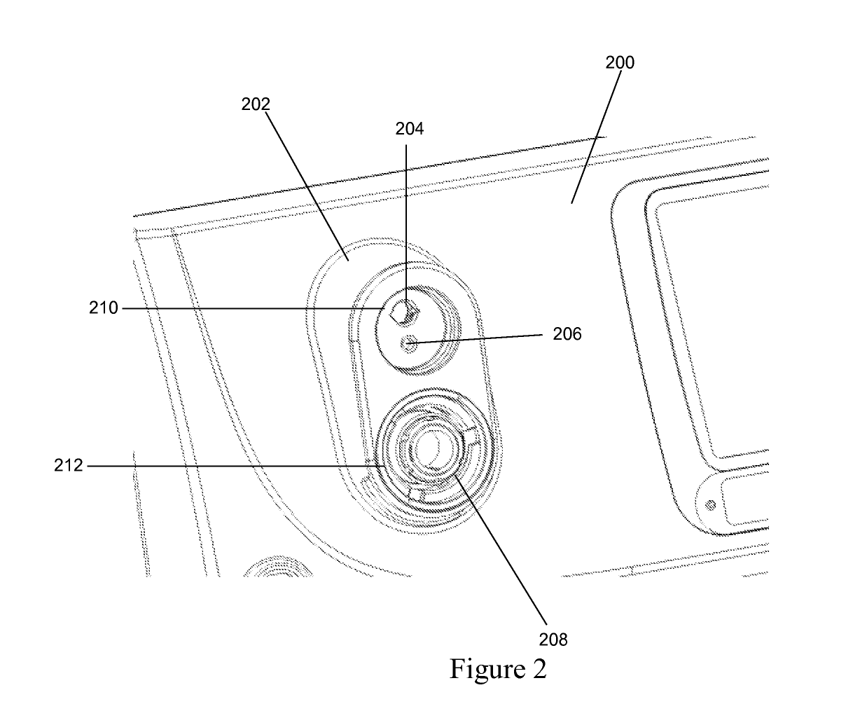

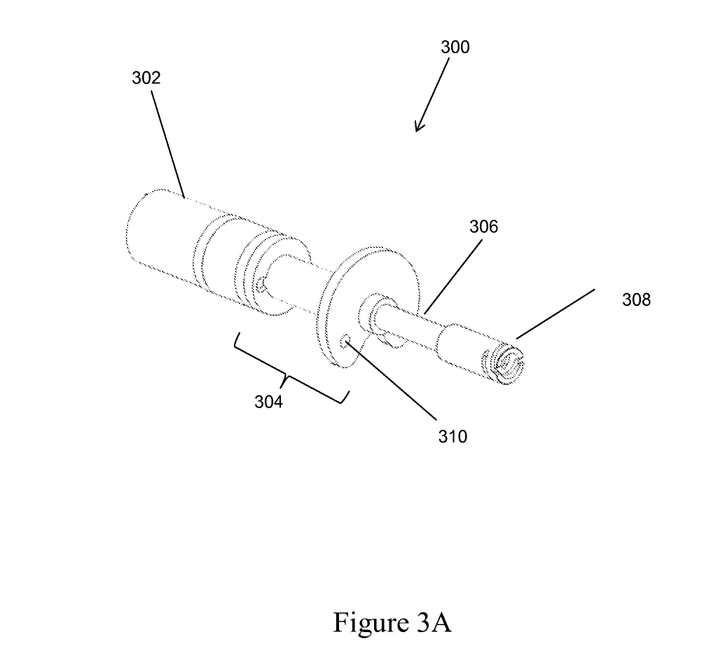

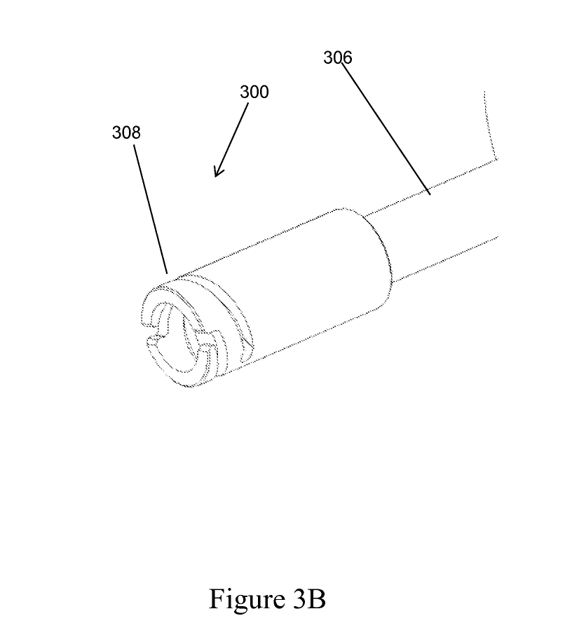

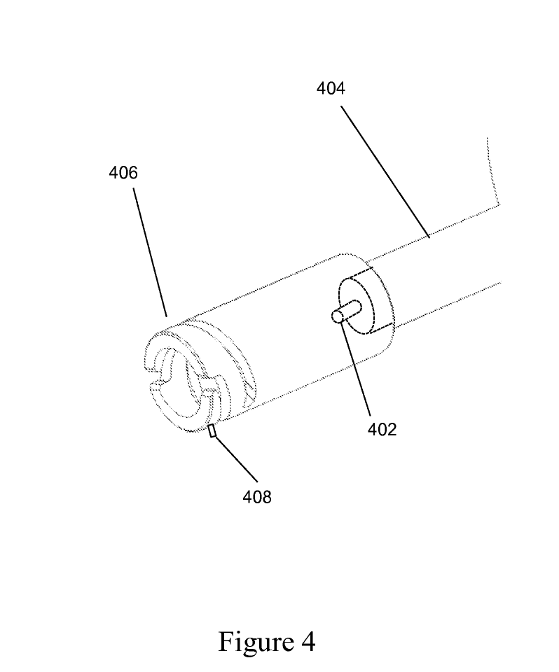

FIG. 2 illustrates a main connector housing on the main control unit front panel, in accordance with an embodiment of the present specification. As shown in FIG. 2, in an embodiment, the main control unit front panel 200 comprises a main connector housing 202 which has two sections, a first section 210 comprising a locking opening 204 and a gas channel opening 206 and a second section 212 comprising a utility cable opening 208. In an embodiment, the gas channel opening 206 receives and connects with the gas channel 110 (shown in FIG. 1A) and the utility cable opening 208 receives and connects with the electric connector 108 (shown in FIG. 1A). In an embodiment, the locking opening 204 houses a receiving structure such as a female bayonet connector coupled with a bayonet motor (shown in FIGS. 3A-3B and 4) and is adapted to receive the locking guide 112 of the endoscope system (shown in FIG. 1A).

FIG. 3A illustrates a bayonet connector, in accordance with an embodiment of the present specification. The bayonet connector 300 comprises a bayonet motor 302 connected to a motor holder section 304, which, in turn, is connected to a bayonet shaft 306, which, in turn, is connected to a receiving structure 308 adapted to receive an elongated protruding member coupled to the main connector of endoscope assembly. In an embodiment, the receiving structure 308 is a female fitting member such as a female bayonet connector. Reference is now made to FIGS. 1A, 2, and 3A, simultaneously. In an embodiment, the bayonet connector 300 is housed within the main control unit 116 (shown in FIG. 1A) such that female bayonet connector 308 is integrally placed within the main connector housing 202 (shown in FIG. 2). In an embodiment, the female bayonet connector 308 is inserted into the locking opening 204 (shown in FIG. 2) of the main connector housing 202 from a back end so that it is capable of receiving the locking guide 112 (shown in FIG. 1A) in order to firmly connect the main connector 102 to the main control unit 116. The motor holder section 304 is designed to aid with the placement of the bayonet connector 300 within the main control unit 116 as the ring portion of holder 304 is provided with openings 310 to screw the bayonet connector 300 into the back portion of the main control unit 116. FIG. 3B illustrates another view of the bayonet connector, in accordance with an embodiment of the present specification, illustrating an exploded view of the bayonet shaft 306 and the bayonet female connector 308.

Referring back to FIG. 1, in an embodiment, the connection of the main connector 102 with the main connector housing 122 is recognized either automatically by using a sensor device or manually by pushing an operation button that may be located on main control unit 116. Upon recognition, the bayonet motor 302 locks the main connector 102 with the main control unit 116.

FIG. 4 illustrates a bayonet shaft, in accordance with an embodiment of the present specification. In an embodiment, a sensor device 402 is provided on an end of bayonet shaft 404 coupling the shaft with the female bayonet connector 406 as shown. When a locking guide is inserted into the female bayonet connector 406, the sensor device 402 is activated and causes the bayonet motor to lock the main connector to the main connector housing. In an embodiment, the sensor device 402 comprises a micro switch which is connected to the bayonet motor 302 (shown in FIG. 3A) positioned in the main control unit. When the locking guide inserted into the female bayonet connector 406, the micro switch 402 is pressed and it transmits a signal which activates the bayonet motor 302.

In another embodiment, a sensor device 408 is provided on an outer tip of the female bayonet connector 406. When the locking guide is inserted into a locking opening of a main connector housing, the sensor device 408 is activated causing the bayonet motor to lock the bayonet female fitting 406 around the locking guide. In the embodiment shown in FIG. 4, while two sensor devices 402 and 408 are shown for exemplary purpose, in an embodiment, only one of these sensor devices can be used.

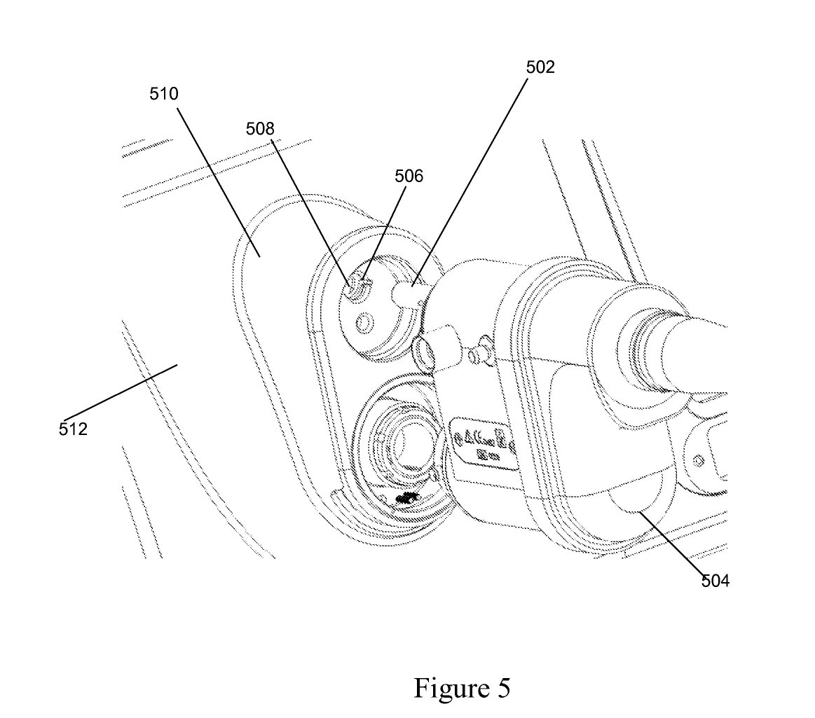

FIG. 5 illustrates a locking guide being inserted into a female bayonet connector, in accordance with an embodiment of the present specification. As illustrated in FIG. 5, an elongated protruding member such as a locking guide 502 of a main connector 504 is being inserted into a female bayonet connector 506 housed within a locking opening 508 of a main connector housing 510 on a main control unit 512.

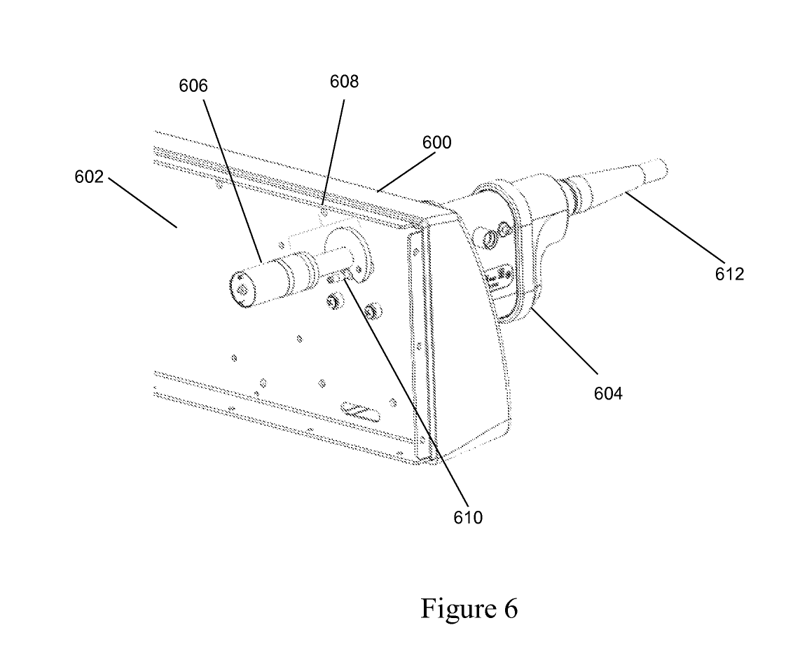

FIG. 6 illustrates a rear view of a main control unit panel when a main connector is connected to a main connector housing, in accordance with an embodiment of the present specification. FIG. 6 shows a rear panel 602 of a main control unit 600. A main connector 604 is connected to the main connector housing of the main control unit 600 by inserting a locking guide of the main connector 604 into a female bayonet connector, as illustrated in FIG. 5. In an embodiment, a bayonet motor 606 and motor holder 608 coupled with a bayonet shaft with a bayonet female connector at one end protrudes, as illustrated in FIG. 6, from the rear panel 602 of the main control unit 600. A gas channel 610 of the main connector 604 is inserted into a gas channel opening of the main connector housing and protrudes from the rear panel 602 as illustrated. The distal end of the main connector 604 is coupled with a utility cable 612.

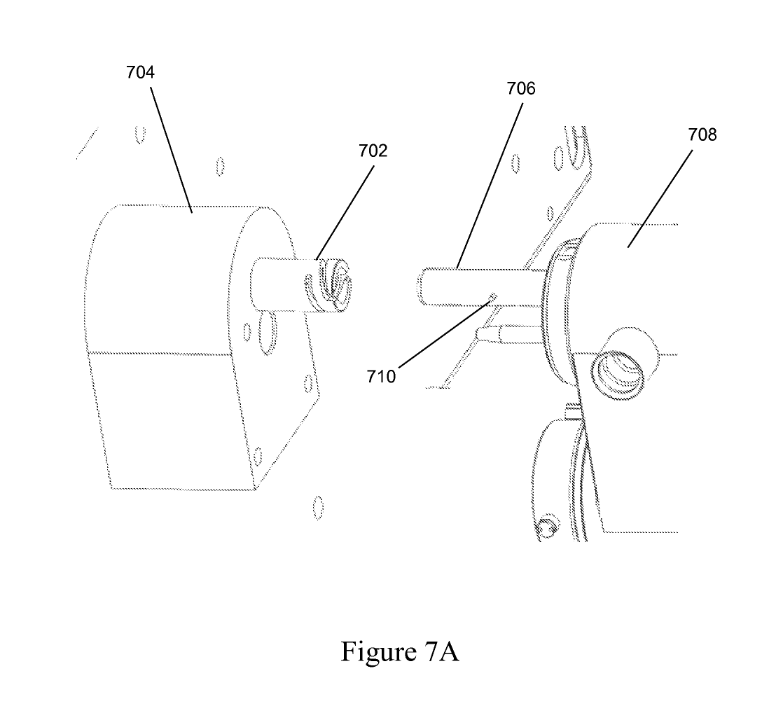

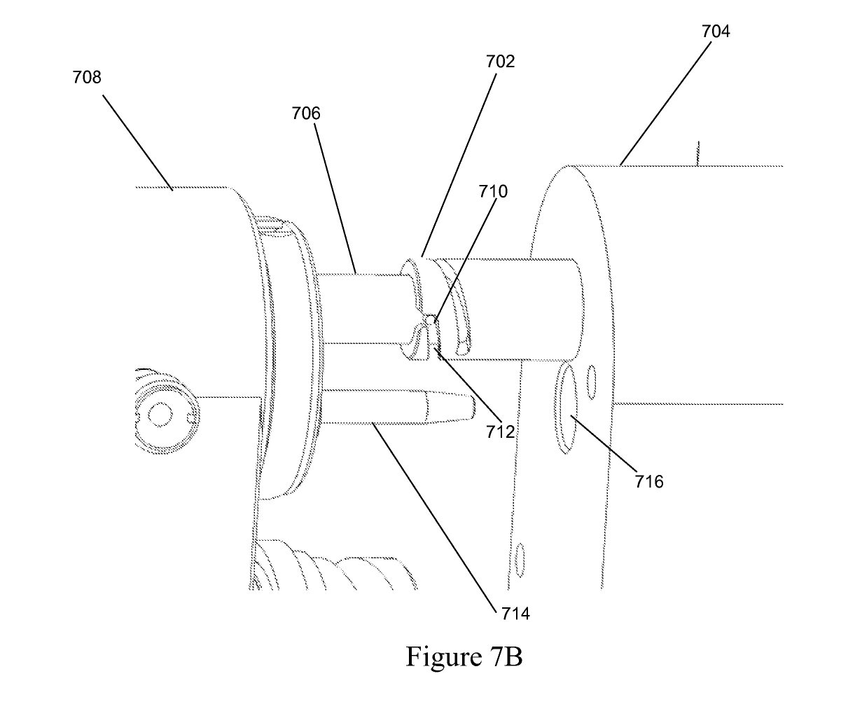

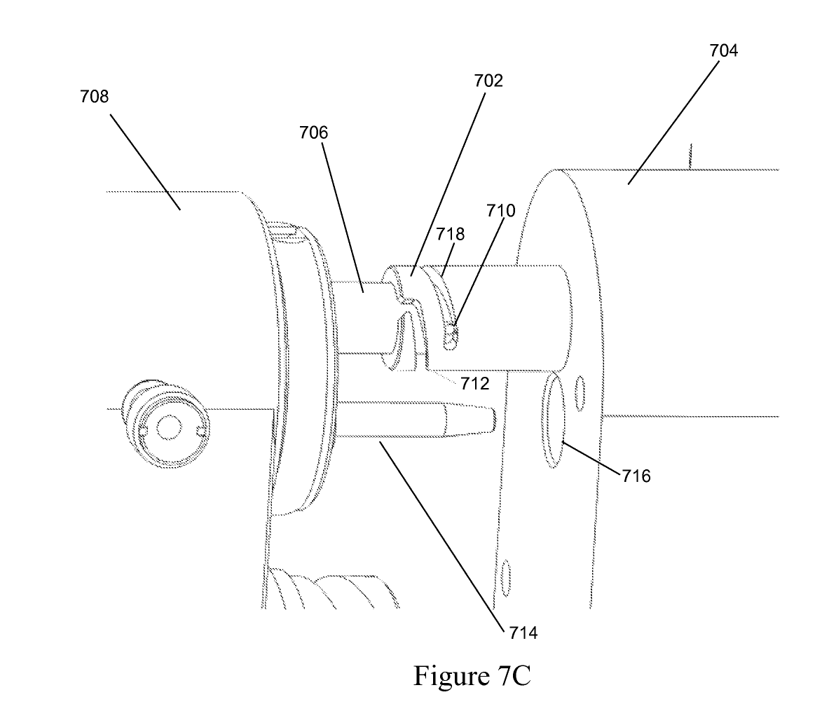

FIG. 7A illustrates a female bayonet connector positioned over a locking opening in a main connector housing and the main connector comprising a locking guide, in accordance with an embodiment of the present specification. As illustrated, a receiving structure such as a female bayonet connector 702 is housed within a locking opening of the main connector housing 704. An elongated protruding member such as a locking guide 706 coupled with a main connector 708 can be inserted into the female connector 702 for securely connecting the main connector 708 to the main connector housing 704. The locking guide 706, in an embodiment, is a guide rod of the main connector 708. A protruding pin 710 is coupled with the locking guide 706 in a transverse plane as illustrated and enables locking of the main connector 708 to the main connector housing 704, when the locking guide rod 706 is inserted into the bayonet female fitting 702.

FIG. 7B illustrates a locking guide placed at the entrance of a female bayonet connector housed within a locking opening in a main connector housing, in accordance with an embodiment of the present specification. As illustrated in FIG. 7B, when the locking guide rod 706, of the main connector 708, is placed at the entrance of the female bayonet connector 702 the protruding pin 710, present on the locking guide 706, is positioned into and locks into a first portion 712 of a contiguous groove cut into the front end of the female bayonet connector 702. FIG. 7B also shows a gas channel 714 which is inserted in the gas channel opening 716 present on the main connector housing 704, when the main connector is locked with the main control unit.

FIG. 7C illustrates a locking guide securely locked within a female bayonet connecter housed within a locking opening in a main connector housing, in accordance with an embodiment of the present specification. As illustrated in FIG. 7C, in an embodiment, when the locking guide 706 is inserted into the female bayonet connector 702, the protruding pin 710 locks into a second portion 718 of the contiguous groove cut into the front end of the female bayonet connector 702.

In an embodiment, a sensor on female connector 702 activates a bayonet motor which causes the female connector 702 to twist around the locking guide 706 and lock the protruding pin 710 within its grooves, which aids in locking the main connector 708 within the block 704 located within main control unit.

In an embodiment, the sensor device located on the female connector 702 comprises a micro switch which is coupled to the bayonet motor positioned in the main control unit. In an embodiment the micro switch directly transmits a signal to the bayonet motor to activate/deactivate the same. In other embodiment, the micro switch is coupled to control unit/circuit in the main control unit such that the micro switch transmits a signal to said control unit and the control unit in turn regulates the functioning of bayonet motor depending on the signal received from the micro switch.

The present specification describes a bayonet motor coupled to the female bayonet connector mounted on the main control unit to engage/disengage the bayonet connection. In an embodiment, the bayonet motor is a simple DC motor which is powered either by a battery or by the direct power received through the main control unit. The phrase “bayonet motor” has been used to identify the motor because the purpose of this motor is to engage/disengage the bayonet connection as per the system requirement.

In an embodiment, the present specification provides a release mechanism for detaching a main connector from a main control unit. The release mechanism of the present specification reduces the manual physical force needed for pulling out/disconnecting the main connector from the main control unit. In an embodiment, the main connector is released from the locking opening of the main connector housing on the main control unit by discharging or ejecting the locking guide from the female bayonet connector when the power is shut down.

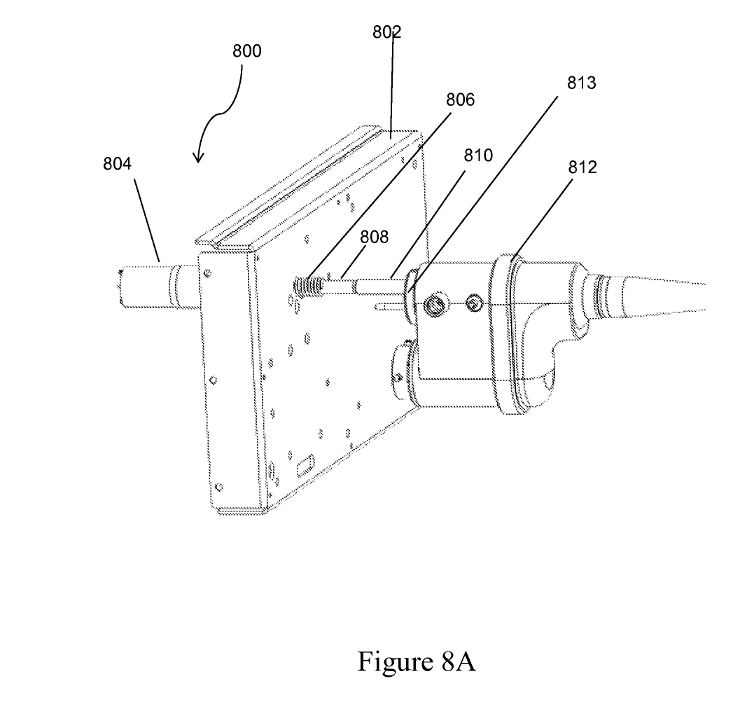

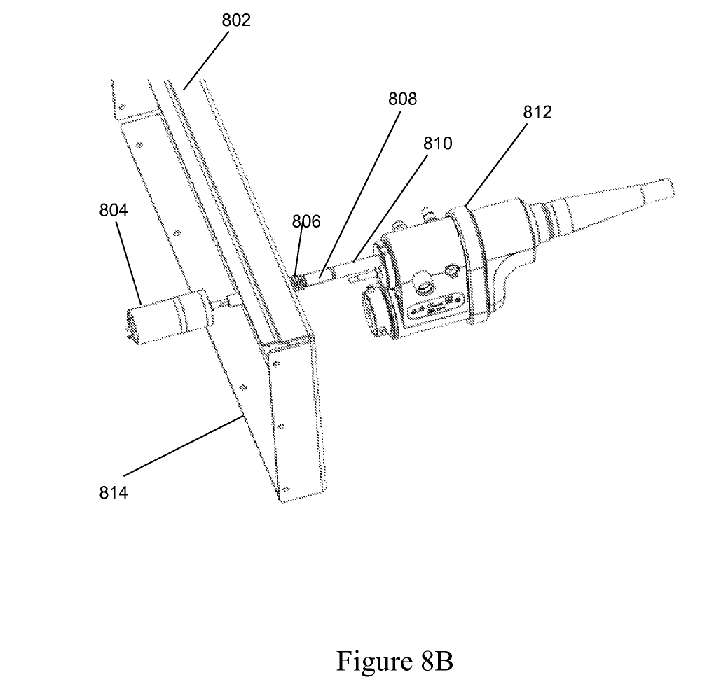

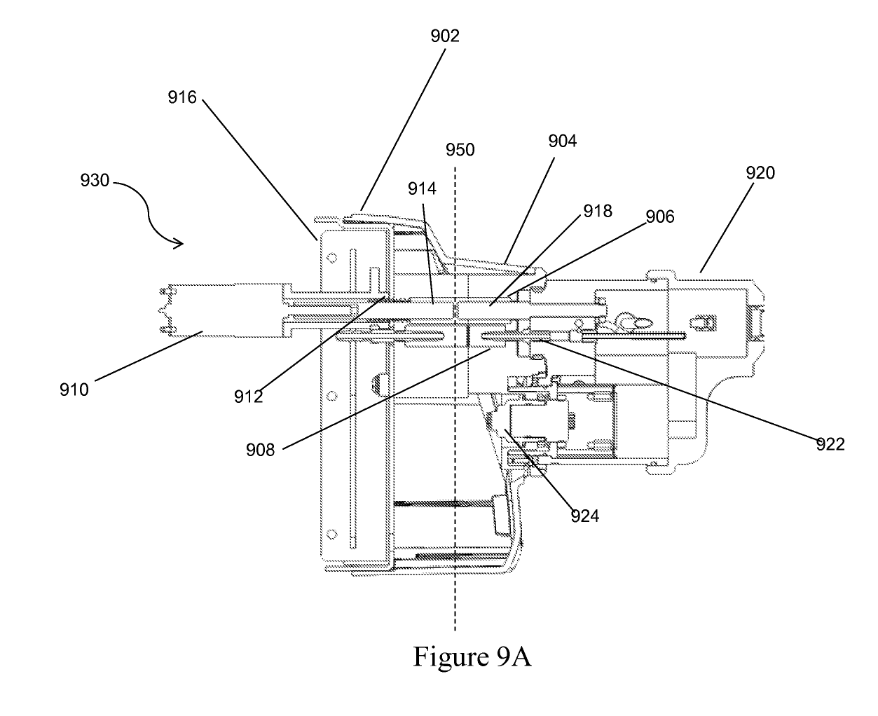

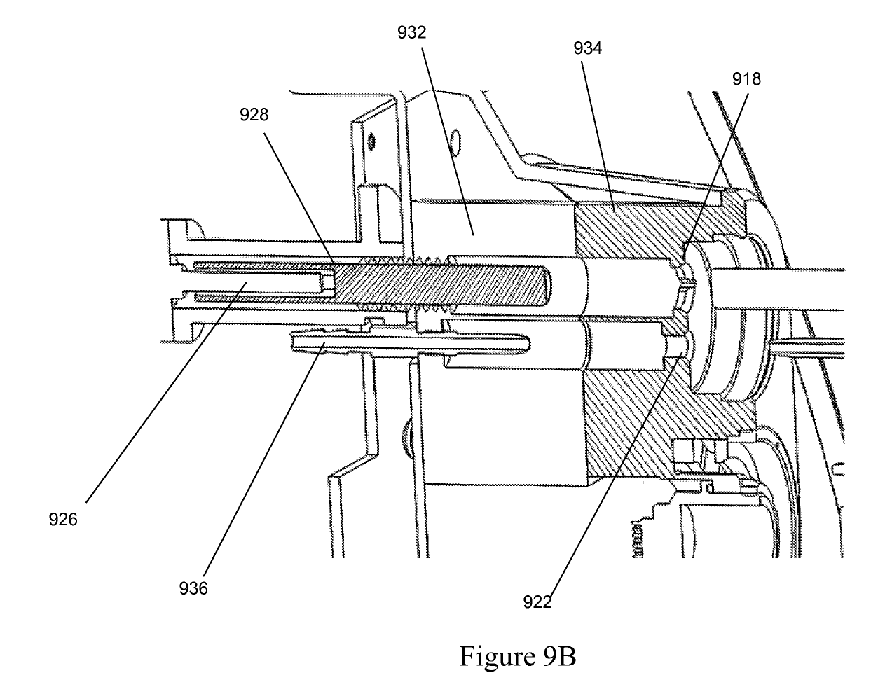

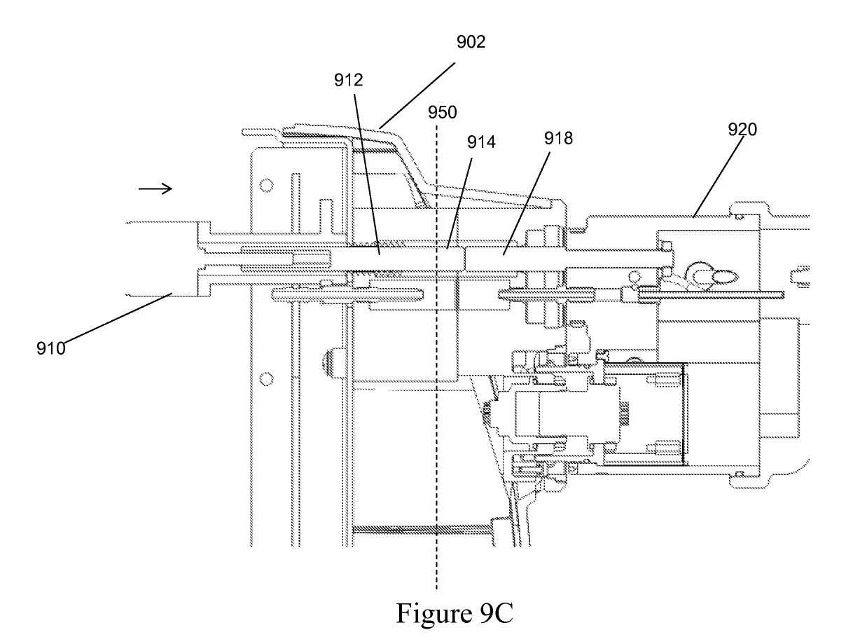

FIGS. 8A, 8B, 9A, 9B, 9C together illustrate another embodiment of the present specification to disengage a main connector from the main control unit of an endoscope. In the above embodiment, a motor and shaft based mechanism is used such that when the shaft is in a first position in the locking opening, the locking guide coupled to the main connector can be inserted and secured in the locking opening. In an embodiment, to release the connection between the main control unit and the main connector, the motor coupled with the shaft is activated such that the motor pushes the shaft to a second position, which in turn pushes out the locking guide from the locking opening of the main control unit and hence releases the connection. In an embodiment, the first position of the shaft is a backward/reverse position and the second position is a forward position.

FIG. 8A illustrates a main connector being released from a main control unit, in accordance with an embodiment of the present specification. FIGS. 8A and 8B illustrate a portion of the main control unit 802 comprising a releasing system 800. In an embodiment, the releasing system 800 comprises a motor 804 coupled with a threaded/protruding shaft 808. In an embodiment the shaft 808 typically having a threaded portion 806 which allows the shaft to move along the axis in forward and backward direction. In an embodiment, when main connector 812 is placed within the main control unit 802, typically before an endoscopic procedure is performed, a locking guide 810 coupled with a main connector 812 is inserted within the main control unit 802 and comes into contact with threaded/protruding shaft 808. The threaded/protruding shaft 814 is in a first or backward position and hence there is optimum space in the locking opening to accommodate the locking guide coupled to the main connector. In an embodiment, a spring ring 813 coupled to the main connector 812 is used to secure the connection between locking guide 810 and the main control unit. In an embodiment, upon pressing an operation button, adapted to release the connector 812 from the main control unit 802, typically at the end of the endoscopic procedure, the motor 804 is activated and it causes the threaded/protruding shaft 808 to move in a forward direction to a second position. The forward movement of the threaded/protruding shaft 808 pushes the locking guide 810 backwards causing the main connector 812 coupled with the locking guide 810 to be disconnected/ejected/released from the main control unit 802.

In an embodiment, the operation button is located on the screen of main control unit 802. In one other embodiment, the button may be located at an easily accessible location on the front panel of the main control unit 802. In another embodiment, the button is located on the main connector 812. FIG. 8B shows an alternate view of the releasing system 800 described in FIG. 8A. As illustrated in FIG. 8B, the back panel 814 of the main control unit 802 is coupled to the motor 804 which aids in releasing the main connector from the main control unit.

FIG. 9A illustrates a cross-sectional view of a main connector connected to a main control unit, in accordance with an embodiment of the present specification. As illustrated the main control unit 902 comprises a main connector housing 904 comprising a locking opening 906, a gas channel opening 908 and a utility cable opening adapted to be coupled with an endoscope electric connector 924. The locking system 930 is placed within the main control unit 902 and comprises a motor 910 coupled with a threaded/protruding shaft 914 which is fitted into the locking opening 906 through a main control unit inside panel 916. In an embodiment the shaft 914 typically having a threaded portion 912 which allows the shaft to easily move along the axis in forward a backward direction. In an embodiment, the shaft 914 is D-shaped and the corresponding hole in the locking opening which accommodates the shaft 914 is also D shaped. The D-shaped configuration allows the shaft 914 to move longitudinally inside the locking guide opening and push locking guide during the release action illustrated in FIG. 8A. When the locking guide 918 is inserted into the locking opening 906, the threaded/protruding shaft 914 is in a backwards/‘reverse’ position allowing locking guide 918 to be secured inside the main control unit 902. There could be multiple methods to secure the locking guide 918 inside the main control unit 902. In an embodiment, a spring ring connected to the locking guide 918 is used to secure the connection. A gas channel 922 of the main connector 920 fits inside the gas channel opening 908 of the main control unit 902. An electric portion 924 of the main connector 920 fits into a utility cable opening on the main control unit 902. Broken line 950 illustrates the position of threaded/protruding shaft 914 when the locking guide 918 is inserted into the locking opening 906 and the connector 920 is connected with the main control unit 902.

FIG. 9B illustrates a cross-sectional view of another embodiment of the present specification showing a main connector connected to a main control unit. In the above embodiment, a D-shaped shaft 926, that has one curved surface and one flat surface, is shown as inserted into a D-shaped hole 928 (configured for receiving the D-shaped shaft) from the back side in the locking opening of the main control unit. The locking guide 918 and the gas channel 922 of the main connector are shown as fully inserted in the corresponding openings in the main control unit. In an embodiment, 934 and 932 are the connecting portions in the main control unit which aid in firmly holding the locking guide 918 and the gas channel 922 and also provide gas to the gas channel 922 through a pump (not shown) in the main control unit which is connected to the nozzle 936.

FIG. 9C illustrates a cross-sectional view of a main connector being released from a main control unit, in accordance with an embodiment of the present specification. For releasing the main connector 920 from the main control unit 902 with the application of minimal physical force, the motor 910 is activated which causes the threaded/protruding shaft 914 to move in a forward direction as illustrated. The forward movement of the threaded/protruding shaft 914 pushes the locking guide 918 backwards causing the main connector 920 coupled with the locking guide 918 to be disconnected/ejected/released from the main control unit 902. The position of the threaded/protruding shaft 914 relative to the position of broken line 950 illustrates the movement of threaded/protruding shaft 914 from a first backward position to a second forward position when the main connector is disengaged from the main control unit

In an embodiment, the motor 910 is activated by pressing an operations button located on the main control unit or the endoscope assembly.

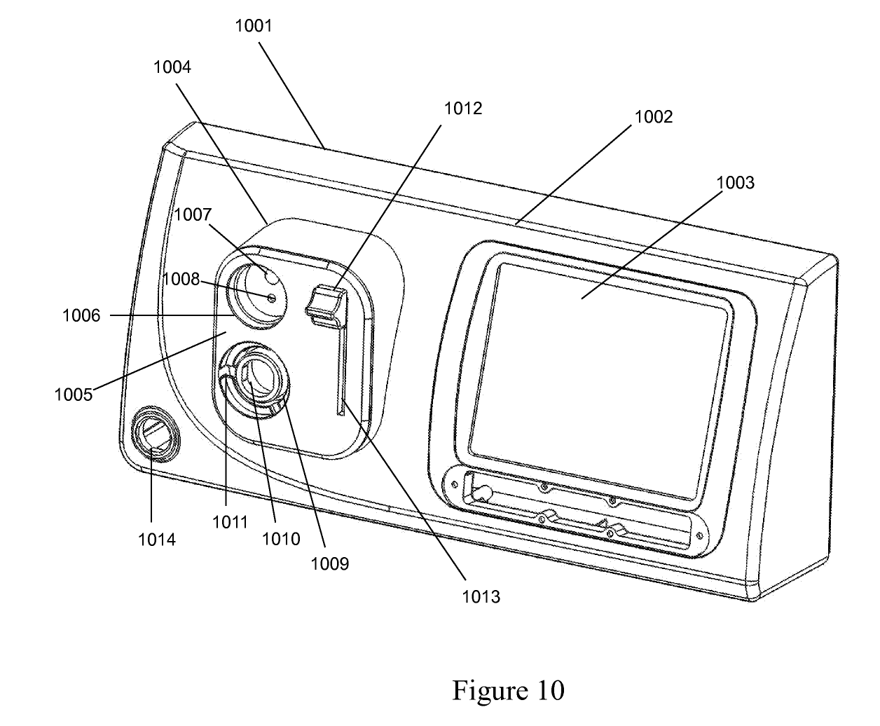

FIG. 10 illustrates a system for connecting a main connector with a main control unit by using a bayonet connection mechanism and a crankshaft arrangement in accordance with an embodiment of the present specification. In the embodiment shown in FIG. 10, the main control unit 1001 comprises a front panel 1002 having a screen 1003 for displaying operational information concerning an endoscopy procedure when the endoscope is in use. Main control unit 1001 further comprises a main connector housing 1004 having a front panel 1005. The main connector housing front panel 1005 comprises a first section 1006 containing the locking/light guide opening 1007 and gas channel opening 1008 and a second section 1009 comprising a utility cable opening 1010. The locking guide opening 1007 and gas channel opening 1008 are configured to receives and connect with the locking guide and gas channel respectively present on the main connector and the utility cable opening 1010 is configured to receive and connect with the electric connector of scope. The switch 1014 is used to switch on or switch off the main control unit.

Section 1009 of the main connector housing 1004 comprises a female bayonet connector 1011, mounted over the utility cable opening 1010. In an embodiment, the female bayonet connector 1011 has a cylindrical section with L-shaped slots which are configured to receive and connect with the male bayonet connector attached to the main connector of the scope. In an embodiment, the male bayonet component located on the main connector comprises a cylindrical section with a plurality of radial pins which are inserted into the L-shaped slots on the female bayonet connector.

The main connector housing 1004 also comprises a slidable switch 1012 which slides vertically through a slider groove 1013. The slidable switch 1012 and the slider groove 1013 are connected on one end of an internally positioned crankshaft. The other end of the crankshaft is connected to the cylindrical section of the female bayonet connector enabling the translation of the linear motion of the slidable switch into the rotational motion of the female bayonet connector.

As shown in FIG. 10, the slidable switch 1012 is in a first position. In a first position, in an embodiment, the slidable switch is open and correspondingly, the cylindrical section of the female bayonet connector 1011 is in an unlocked position. In an embodiment, when the main connector of the scope is inserted into the main connector housing 1004 of the main control unit 1001, the radial pins located on male bayonet component mounted on the main connector of the scope are inserted into the female bayonet connector 1011. Subsequently, the slidable switch 1012 is moved vertically downward through slider groove 1013 and via the crankshaft mechanism attached to the slidable switch, the linear motion is translated into a rotational motion, causing the cylindrical section of the female bayonet connector 1011 to move in a circle and lock with the male bayonet component of the main connector. Once the bayonet connection is complete, the slidable switch is in a second, closed position and the endoscope is activated and ready for use. The embodiment described in the present specification also provides a very convenient mechanism to disconnect the main connector with the main control unit with application of minimal physical force. In an embodiment, after the procedure is finished, the user can move the slidable switch 1012 vertically upward, which causes the female bayonet connector to move in a circular direction (in the opposite direction of movement when the switch is moved downward) and unlock the male bayonet component and thereby release the main connector from the main control unit.

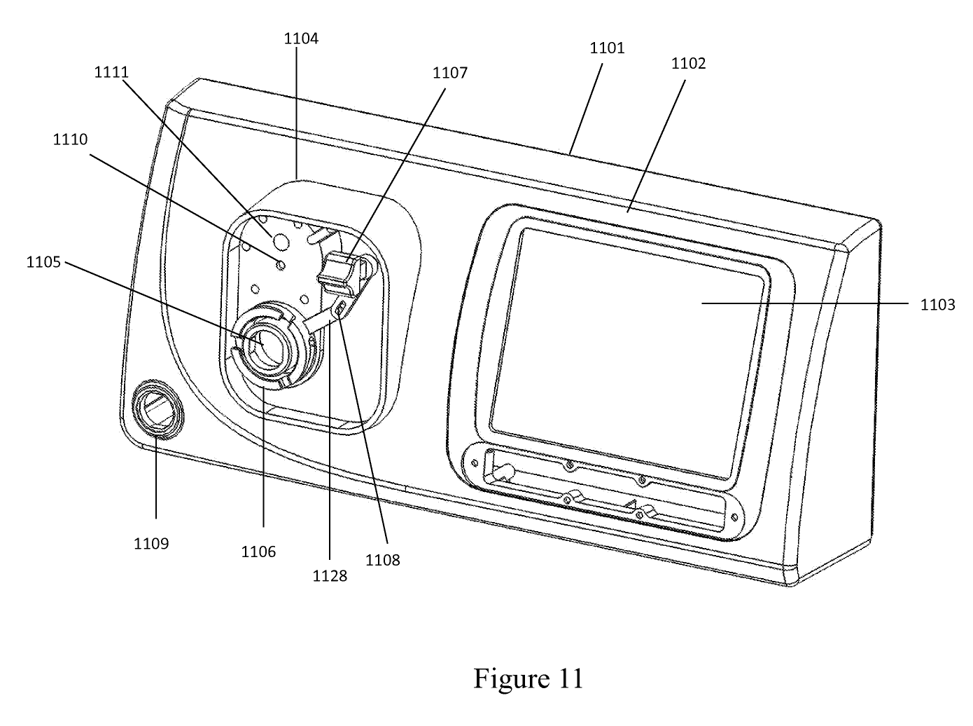

FIG. 11 shows the main control unit without the outer panel of main connector housing and with the slidable switch in a first position in accordance with an embodiment of the present specification. As shown in FIG. 11, the main control unit 1101 comprises a front panel 1102, which contains a display unit 1103 and a main connector housing 1104. The main connector housing 1104 comprises a utility cable/main connector opening 1105 and a female bayonet connector 1106 mounted over the surface of utility cable opening 1105. The female bayonet connector 1106 has a cylindrical section with a plurality of L-shaped slots which are configured to receive and connect with the male bayonet component attached to the main connector of endoscope assembly. The main connector housing 1104 also comprises a gas channel opening 1110 and a locking/light guide opening 1111 which are configured to receive and connect with the gas channel and locking/light guide pin on the main connector of endoscope assembly.

The female bayonet connector 1106 is connected to a crankshaft 1108 through a connecting rod 1128. The crankshaft 1108 is in turn connected to slidable switch 1107 which can move vertically up or down through a slider groove (not shown). Switch 1109 is used to switch on or switch off the main control unit 1101. In the embodiment shown in the FIG. 11, the slidable switch 1107 is in a first position and it is connected to the female bayonet connector 1106 through a crankshaft 1108. In the first position, in an embodiment, the slidable switch is open and therefore, the cylindrical section of the female bayonet connector 1106 is unlocked. The male bayonet component of the main connector docks with the female bayonet connector 1106 when the main connector is placed into the main control unit 1101. Subsequently, the slidable switch 1107 is moved in a downward direction and, the crankshaft 1107 translates this vertical movement into a rotational motion of the cylindrical section of the female bayonet connector 1106, which causes it to lock with the male bayonet component of the main connector of the scope. In an embodiment, the radial pins located on the cylindrical section of the male bayonet component completely fit into the L-shaped grooves located in the female bayonet connector 1106 when the bayonet connection is complete and the slidable switch has reached a second, closed position.

There could be multiple ways in which the crankshaft 1108 is connected to the female bayonet connector 1106 and slidable switch 1107. In an embodiment, the crankshaft 1108 is connected with help of forged connecting rods permanently fixed to the female bayonet connector 1106 and the slidable switch 1107. In one alternative embodiment, the various components in the structure are connected using screws or other connection means.

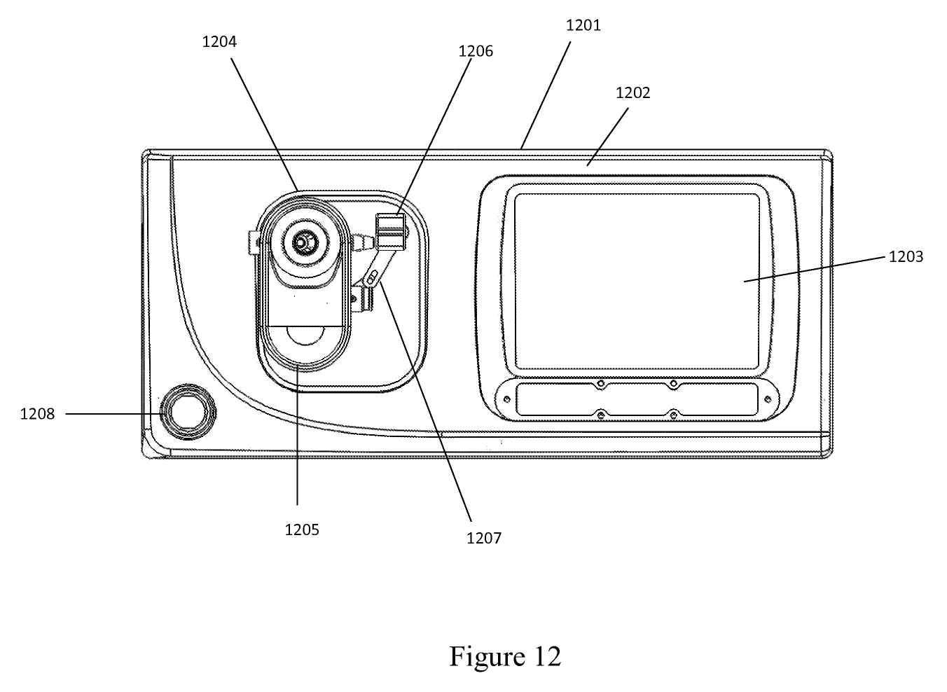

FIG. 12 illustrates a two-dimensional front view of the main control unit without the outer panel of the main connector housing, connected to the main connector, and with a slidable switch in a first position in accordance with an embodiment of the present specification. As illustrated in FIG. 12, the main control unit 1201 comprises a front panel 1202, which contains a display screen 1203, a main connector housing 1204 and a switch 1208 adapted to switch on or switch off the main control unit 1201. The main connector 1205 is placed in the main connector housing 1204 and the slidable switch 1206 is shown in a first position and is connected to a crankshaft 1207. In an embodiment, the first position of slidable switch 1206 corresponds to an open position which means the cylindrical section of the female bayonet connector on main control unit 1201 is in an unlocked position. In the embodiment shown in FIG. 12, the male bayonet component of the main connector 1205 is docked with the female bayonet component located over the utility cable opening in the main control unit 1201. In this embodiment, when the slidable switch 1206 will be moved from first position in a vertical downward direction, the cylindrical section of female bayonet component will start rotating and shall connect with the male bayonet component.

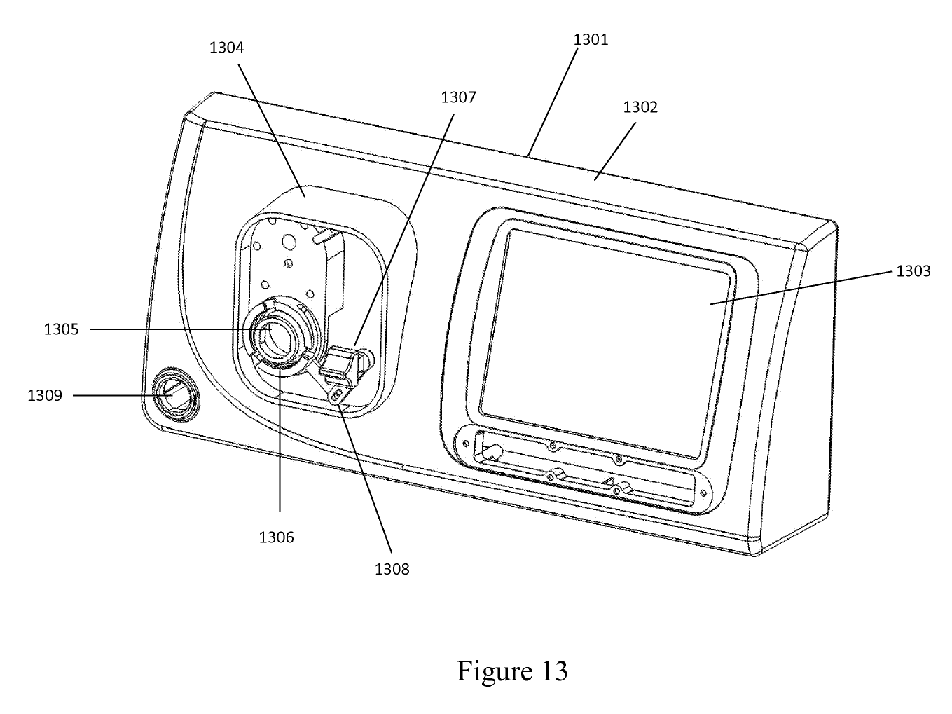

FIG. 13 shows the main control unit without the outer panel of main connector housing and with the slidable switch in a second position in accordance with an embodiment of the present specification. As illustrated in FIG. 13, the main control unit 1301 comprises a front panel 1302, which contains a display unit 1303 and a main connector housing 1304. 1309 represents the on/off switch for the main control unit 1301. The main connector housing 1304 comprises a utility cable opening 1305 and a female bayonet connector 1306 mounted over the surface of utility cable opening 1305. The female bayonet connector 1306 is connected to a crankshaft 1308 through a connecting rod. The crankshaft 1308 is in turn connected to slidable switch 1307 which can move vertically up or down through a slider groove (not shown). In FIG. 13, the slidable switch 1307 is shown in a second position. In a second position, in above embodiment, the slidable switch is in a closed position and the corresponding cylindrical section of the female bayonet connector 1306 is in a locked position. The above figure is only for illustration purpose. In an embodiment, the slidable switch 1307 is moved to second, closed position only after placing the main connector in the main connector housing 1304. In an embodiment, the main connector can be unlocked by moving the slidable switch 1307 in an upward direction. The system disclosed in the above embodiment, translates the vertical linear motion of the slidable switch 1307 into circular motion of the female bayonet connector 1306 which in turns unlocks the main connector from the main control unit.

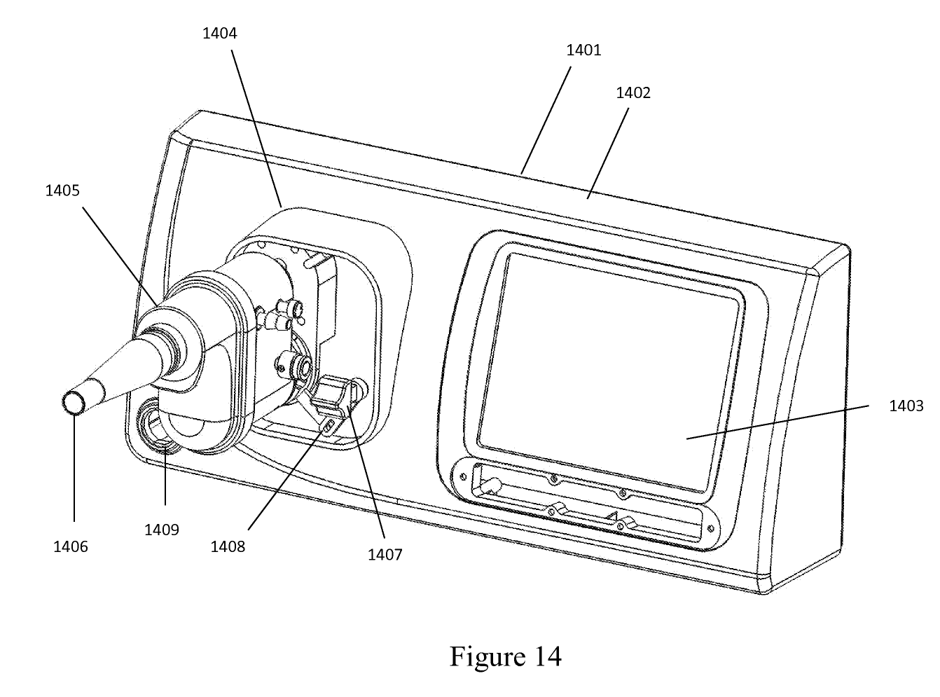

FIG. 14 depicts an embodiment of the main control unit without the outer panel of the main connector housing and with the main connector connected to the main control unit and slidable switch in a second position. As shown in FIG. 14, the main control unit 1401 comprises a front panel 1402 which contains a display screen 1403, a main connector housing 1404 and a switch 1409 which represents the On/Off switch of the main control unit 1401. The main connector 1405 is attached to the handle of the scope through utility cable 1406 at one end and it connects to the main control unit 1401 through its other end. As shown in FIG. 14, the main connector 1405 is housed inside the main connector housing 1404 and the slidable switch 1407 is in a second position. In a second position, in above embodiment, the slidable switch is in a closed position and the corresponding cylindrical section of the female bayonet connector in the main control unit 1401 is in a locked position. In this embodiment, the male bayonet component (not shown) of the main connector 1405 is locked up with the female bayonet connector (not shown) mounted over the utility cable opening (not shown) of the main connector housing 1404. The male bayonet connector mounted on main connector 1405 comprises a cylindrical section with a plurality radial pins that fit into the L shaped grooves present on the female bayonet connector. Once the male bayonet connector on main connector 1405 came in touch with female bayonet connector in main connector housing 1404, the slidable switch 1407 was moved vertically down which locked the male and female bayonet connectors with the help of a crankshaft 1408. The system disclosed in the present specification translates the vertical motion of the slidable switch 1407 into rotational motion of the female bayonet connector in main connector housing 1404, with the help of a crankshaft 1408. Once the user slides down the slidable switch 1407 through the slider groove, the cylindrical section of female bayonet connector, mounted over the utility cable opening in main connector housing 1404, moves in a circular direction thereby letting the radial pins on male bayonet connector of main connector 1405 to fit properly in its L-shaped grooves. This causes the main connector to lock with the main control unit and activates the various functionalities in endoscope. In above embodiment, the use of crankshaft 1408 makes it very convenient for the user to lock or unlock the bayonet connecters. Further, the use of bayonet connector ensures that the connection between main control unit and the main connector is reliable and secure which is very important before using the endoscope.

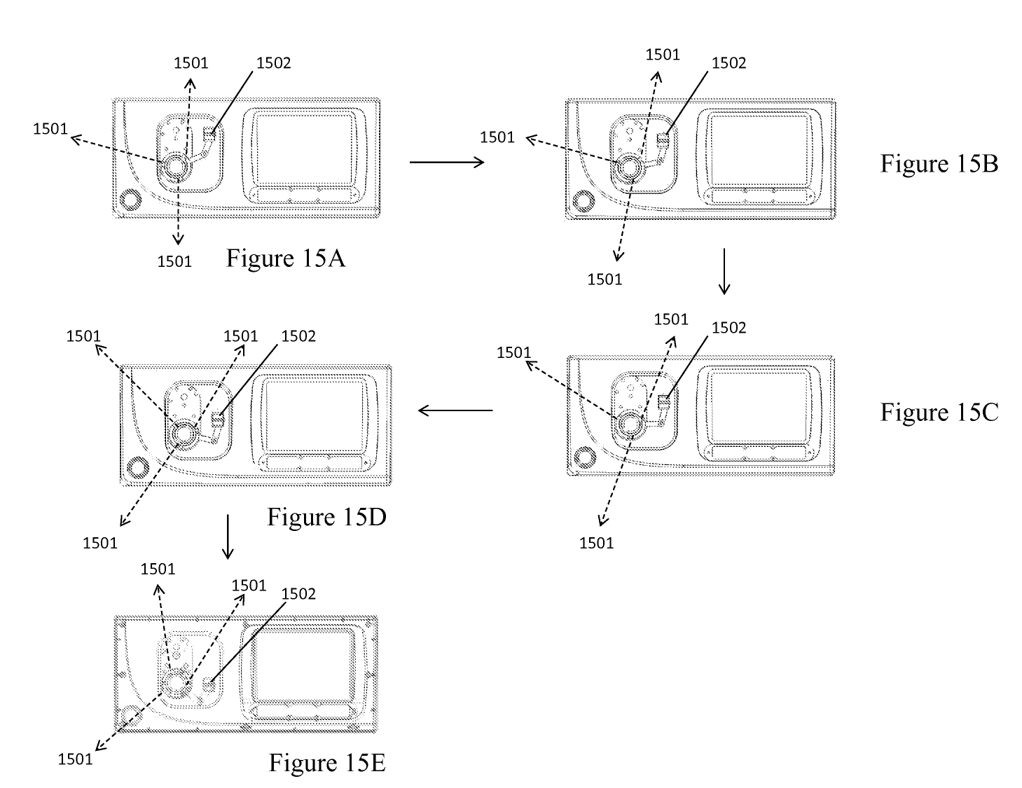

FIGS. 15A to 15E illustrate the movement of female bayonet connector and the crankshaft caused by change in position of slidable switch in accordance with an embodiment of the present specification. As shown in the first stage in FIG. 15A, 1501 represent the grooves in female bayonet connector which are configured to receive and connect with the male bayonet component on main connector. At the first stage in FIG. 15A, the slidable switch 1502 is shown in an unlocked position. In an embodiment, as the slidable switch 1502 is moved vertically downward, the cylindrical section of the female bayonet connector moves in a clockwise direction. As shown in the second stage in FIG. 15B, when the slidable switch 1502 has been moved a little bit lower, the position of grooves in the female bayonet connector has also changed accordingly as represented by 1501. Similarly, the third and fourth stages in FIGS. 15C and 15D, respectively, depict two more interim positions of the slidable switch 1502 and the grooves 1501 when the slidable switch is being moved from unlocked or open position to the closed position. The fifth stage in FIG. 15E illustrates the final position when slidable switch 1502 has reached a locking position and accordingly the cylindrical section of female bayonet component containing the grooves 1501 has moved to a position wherein the system can lock the male bayonet component firmly, in case the same would have been inserted initially. For illustration purposes, in FIGS. 15A to 15E, the main connector is not shown to be inserted in the main control unit. Usually the slidable switch 1502 is moved down to a locked position after inserting the main connector in the main control unit. It should be noted that although FIGS. 15A to 15E illustrate the movement of the slidable switch and crankshaft in stages, in operation, the movement is fluid and can be accomplished by sliding the switch fully in one step. In another embodiment, as the slidable switch 1502 is moved vertically downward, the cylindrical section of the female bayonet connector moves in a counterclockwise direction.

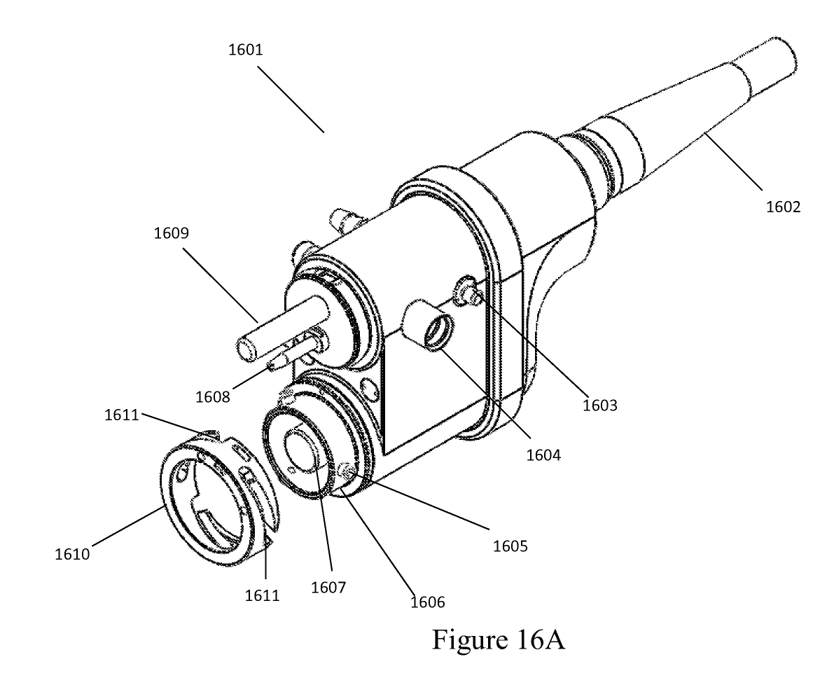

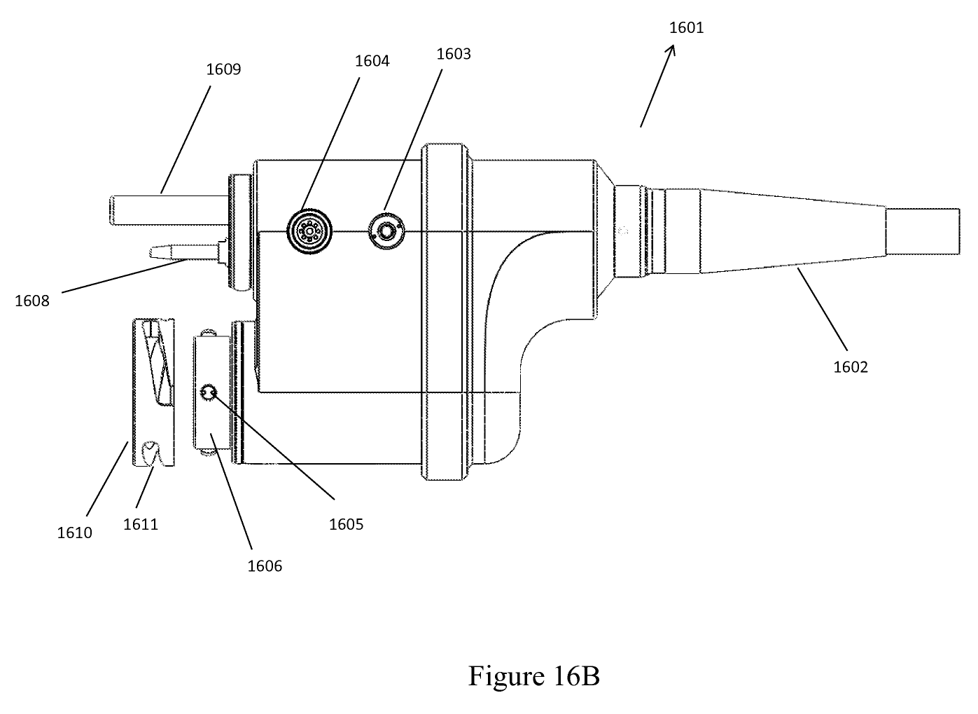

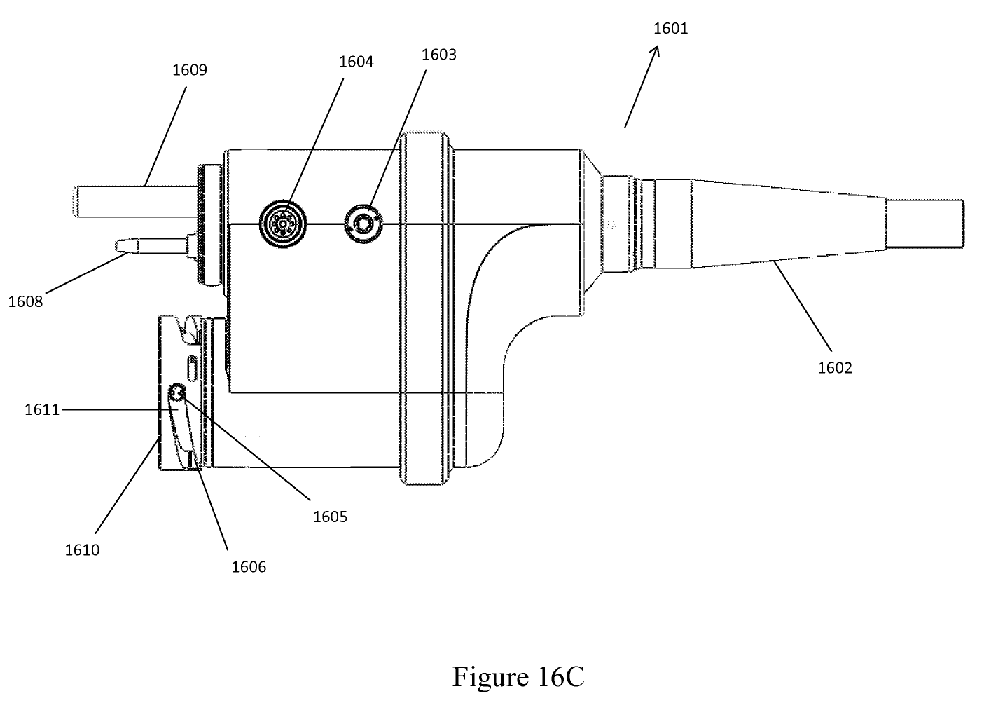

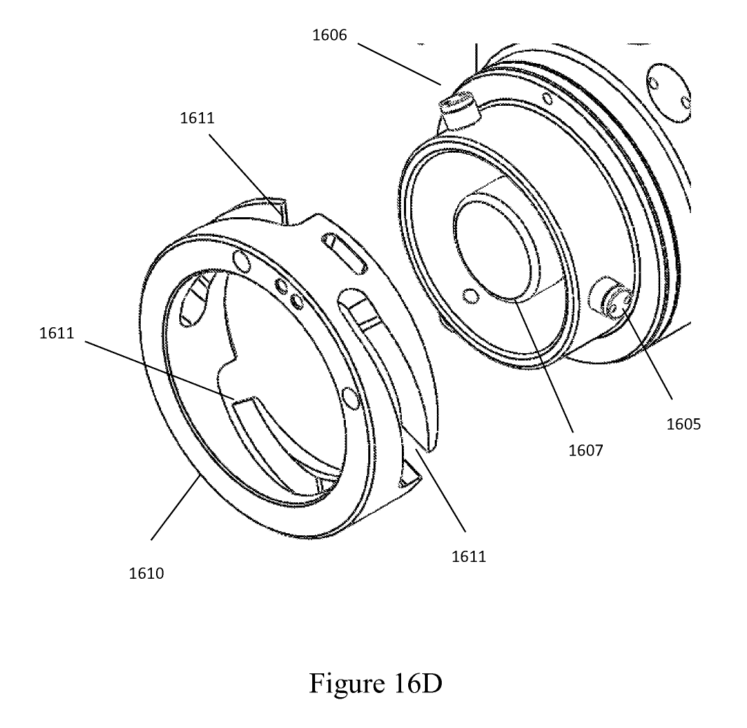

FIG. 16A illustrates a main connector and the female bayonet component separately as per an embodiment of the present specification. As shown in FIG. 16A, a main connector of the endoscope assembly such as main connector 1601 is attached to the handle (not shown) of the endoscope through utility cable 1602 on its one end. As illustrated, the main connector 1601 comprises a jet connector 1603, wherein the jet connector 1603 is typically connected to a fluid supplier to provide fluid to a jet opening in an endoscope tip, a water bottle connector 1604, wherein the water bottle connector 1604 is typically engaged to a water supplier, such as a water bottle or hospital facilities, to provide fluid to an insufflation and/or irrigation system placed within the endoscope tip, an electric connector 1606, wherein the electric connector 1606 connects between electronics components within the endoscope, such as but not limited to, sensors, illuminators, handle of the endoscope and the main control unit to provide electricity to the various components, a gas channel 1608, wherein the gas channel 1608 typically provides gas flow to the tip of the endoscope and a locking guide 1609, wherein the locking guide 1609 typically provides the channel for light source placed in the main control unit in case of fiber optic based light illumination system. The electric connector 1606 comprises at least one electric connector pin 1605 placed on the periphery of electric connector 1606 that fits into the corresponding grooves in a female bayonet connector 1610 and an electric internal male connector 1607 that interfaces with bayonet female connector 1610, and provides for an electric connection between main control unit and the scope. For illustration purpose, the female bayonet connector 1610 is shown separately, however, in an embodiment of the present specification, the female bayonet connector is actually mounted on the surface of main control unit and is coupled to a slidable switch through a crankshaft system. The female bayonet connector 1610 comprises a plurality of L shaped grooves 1611 in which a plurality of electric connector pin 1605 are received and locked with the help of crankshaft system.

FIG. 16B illustrates a side view of the main connector and the female bayonet connector separately in accordance with an embodiment of the present specification. As shown in the FIG. 16B, the main connector 1601 comprises the male bayonet component comprising the electric connector 1606 and a plurality of electric connector pins 1605. The pins 1605 are structured to fit into the L shaped grooves 1611 in the female bayonet component 1610. In an embodiment, the female bayonet component 1611 is present on the surface of main control unit in main connector housing and is attached to a slidable switch through a crankshaft system.

FIG. 16C illustrates a side view of the main connector wherein the male bayonet component is engaged with a female bayonet component in accordance with an embodiment of the present specification. As illustrated in FIG. 16C, the electric pins 1605 are fitted into the L shaped grooves 1611 present in the female bayonet component 1610. In an embodiment of the present specification, the electric connector pin 1605 are fitted into the grooves 1611 by rotating the female bayonet connector 1610 with the help of a slidable switch which is coupled to the female bayonet connector 1610 through a crankshaft system. The crankshaft system allows the user to rotate the female bayonet connector 1610 with application of minimum force.

FIG. 16D illustrates an enlarged view of the female bayonet component present on the main control unit and the male bayonet component present on the main connector in accordance with an embodiment of the present specification. As shown in FIG. 16D, the female bayonet component 1610 comprises a plurality of L shaped grooves 1611. One of ordinary skill in the art could appreciate that while in FIG. 16D, three L shaped grooves are shown, the number of L shaped grooves can lower or higher than three in various embodiments of the present specification. Also, the embodiments disclosed in present specification are not limited to only L shape grooves. The shape of grooves can be different from L shape in embodiments of the present specification. The electrical connector 1606 contains a plurality of electric connector pins 1605 and an electric internal male connector 1607 that interfaces with the female bayonet connector 1610.

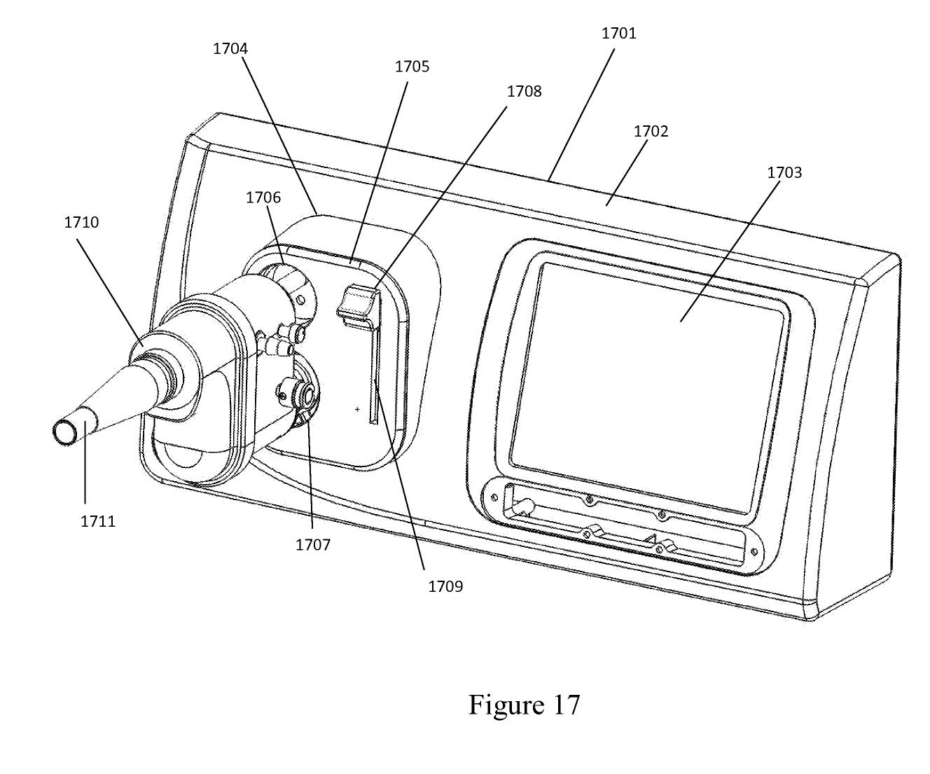

FIG. 17 illustrates an embodiment of the system disclosed in present specification, with the main connector housing covered with its outer panel and with main connector of the scope partially inserted into the main control unit and with the slidable switch in a first position. As shown in FIG. 17, main control unit 1701 comprises a front panel 1702 which contains a display unit 1703 and a main connector housing 1704. The front panel 1705 of the main connector housing 1704 comprises a first section 1706 and a second section 1707. The first section 1706 comprises a locking guide opening and a gas channel opening and the second section 1707 comprises a utility cable opening with a female bayonet connecter mounted over the surface of utility cable opening. The main connector housing 1704 further comprises a slidable switch 1708 and a slider groove 1709 through which the slidable switch 1708 can be made to slide. As shown in FIG. 17, the main connector 1710 is shown partially inserted in the main connector housing 1704. The main connector 1710 is attached to the utility cable 1711 of the endoscope at one end and it connects to the main control unit through its other end. It comprises a male bayonet connector (not shown in figure) which connects with the female bayonet connector mounted over the utility cable opening in section 1707 of the main connector housing 1704. In above embodiment, in first position, the slidable switch 1708 is in an open position and the bayonet components are not connected. Once the male bayonet connector comes in touch with female bayonet connector after the main connector 1710 is completely inserted into the main connector housing 1704, the user can move the slidable switch 1708 vertically downward, which locks the two bayonet components and activates the endoscope assembly.

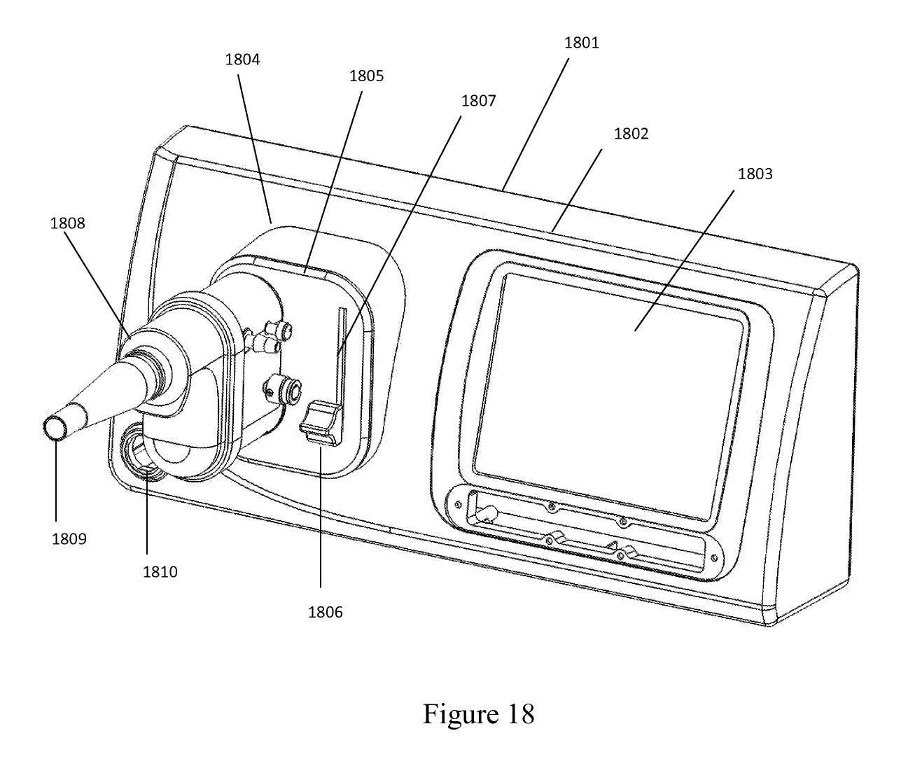

FIG. 18 shows an embodiment of the system disclosed in present specification, with the main connector housing covered with its outer panel and with main connector of the scope inserted into the main control unit and with the slidable switch in a second position. As shown in FIG. 18, the main control unit 1801 comprises a front panel 1802 which contains a display unit 1803 and a main connector housing 1804. The switch 1810 is used to switch on/off the main control unit 1801. The front panel 1805 of the main connector housing 1804 comprises the slidable switch 1806 and slider groove 1807 through which the slidable switch can move up or down in a vertical direction. The main connector 1808 is attached to the handle (not shown) of the scope through utility cable 1809 at one end and it connects to the main control unit through its other end. As shown in FIG. 18, in above embodiment, the main connector 1808 is placed inside the main control unit 1801 and the slidable switch in a second position, wherein the bayonet components are connected or locked which means the endoscope is ready for use. The user can deactivate the endoscope assembly by moving the slidable switch 1806 in an upward direction to the first position.

In an embodiment, instead of providing a mechanical system comprising a slidable switch to operate the crankshaft, an electrical motor based mechanism is used to operate the crankshaft system. In above embodiment, the main control unit comprises an electrical motor which is coupled to the crankshaft system such that when the electrical motor is activated it moves the crankshaft which in turn rotates the female bayonet connector to lock/unlock it with the male bayonet connector. In an embodiment, the electrical motor is connected to the crankshaft through a connecting rod and essentially replicates the function of slidable switch such that the user it not required to move the switch up or down but just needs to operate the electrical motor.

There could be multiple methods to activate/deactivate the electrical motor described in above embodiment. In an embodiment, a simple operational switch such as an on/off switch is provided on the main control unit which is used to activate/deactivate the electrical motor. The switch is connected to an electrical motor which, when activated, moves a rod connected to the crankshaft. This, in turn, causes the female bayonet connector to move in a rotational direction and accordingly lock or unlock with the corresponding male bayonet component. The user would simply switch on or switch off the operations switch by pressing the same.

In another embodiment, instead of using an operations switch to activate the electrical motor, the system comprises a sensor device which detects the presence of the male bayonet connector and activates the motor which causes the crankshaft to move. This in turn causes the female bayonet connector to rotate and engage with the male bayonet connector. In an embodiment, the sensor device is positioned on the outer tip of the female bayonet connector. However, in other embodiments, the sensor device could be positioned at other locations in the endoscope system as long as it can detect that the female bayonet component is docked with the male bayonet component and accordingly activates the electrical motor to operate the crankshaft. In the above embodiment, the operations switch would still be required to activate the electrical motor for disengaging the bayonet connection when the main connector has to be removed from the main control unit.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A connection system for connecting a connector to a main control unit of an endoscope, said connector comprising:
an elongated protruding member adapted to fit into a receiving structure positioned on a surface of said main control unit,
wherein said receiving structure is coupled to a motor which, when activated, causes said receiving structure to rotate and engage or disengage with said elongated protruding member.

2. The connection system as claimed in claim 1, wherein said elongated protruding member has an outer periphery and comprises at least one protruding pin on said outer periphery, wherein said at least one protruding pin is adapted to fit into the grooves of said receiving structure when said receiving structure is rotated.

3. The connection system as claimed in claim 1, wherein said elongated protruding member is a locking guide of the main connector.

4. The connection system as claimed in claim 3, wherein said receiving structure is placed in an opening of the main control unit and said opening is for receiving the locking guide of main connector.

5. The connection system as claimed in claim 1, where said motor is coupled to the receiving structure through a motor holding section and a shaft.

6. The connection system as claimed in claim 1, wherein said motor is coupled to a rear portion of said receiving structure.

7. The connection system as claimed in claim 1, further comprising a sensor device which detects a presence of said elongated protruding member and activates the motor.

8. The connection system as claimed in claim 7, wherein said sensor device is positioned on a front section of the said elongated protruding member such that, when the elongated protruding member is inserted into the receiving structure, the sensor device is activated.

9. The connection system as claimed in claim 7, wherein said sensor device is positioned on an outer portion of the receiving structure.

10. The connection system as claimed in claim 1, wherein said receiving structure is a bayonet connector.

11. The connection system as claimed in claim 1, further comprising a switch adapted to activate the motor for disengaging a connection between said receiving structure and said elongated protruding member.

12. A system for disconnecting a connector portion of an endoscope from a control unit, comprising:
an elongated protruding member coupled to the connector portion; and
a control unit having an opening adapted to receive the elongated protruding member coupled to the connector portion, wherein said opening comprises a shaft coupled to a motor, which when activated, is adapted to cause said shaft to move longitudinally and eject said elongated protruding member, thereby disengaging the endoscope from said main control unit.

13. The system as claimed in claim 12, wherein said shaft comprises a threaded portion.

14. The system as claimed in claim 12, wherein said elongated protruding member is a locking guide of the connector portion.

15. The system as claimed in claim 12, wherein said opening is a locking opening of the control unit.

16. The system as claimed in claim 12, wherein said shaft is D-shaped and the opening in the control unit is D-shaped

17. The system as claimed in claim 12, wherein said motor is activated through an operation button, positioned on the control unit.

18. An endoscope having a connection portion for connecting to a control unit, the connector portion comprising a male fitting member which is adapted to fit into a female fitting member placed in an opening on a surface of the control unit, wherein said female fitting member is coupled to a slidable switch through a crankshaft located in the control unit.

19. The endoscope as claimed in claim 18, wherein said female fitting member and the slidable switch are located on a main connector housing on the main control unit.

20. The endoscope as claimed in claim 18, wherein said male fitting member is a bayonet connector.

21. The endoscope as claimed in claim 18, wherein said female fitting member is a bayonet connector.

22. The endoscope as claimed in claim 18, wherein said female fitting member is placed in an opening of the main control unit, and wherein said opening is for receiving a utility cable.

23. The endoscope as claimed in claim 18, wherein said male fitting member comprises an electrical connection portion.

24. The endoscope as claimed in claim 18, wherein said slidable switch is moved linearly in a vertical or horizontal direction.

25. The endoscope as claimed in claim 18, wherein said crankshaft is a forged structure fixedly attached to the female fitting member and/or the slidable switch.

26. The endoscope as claimed in claim 18, wherein said crankshaft is fixedly attached to said female fitting member and/or said slidable switch using screws.

27. The endoscope as claimed in claim 18, further comprising a slider groove over which the slidable switch can slide.

28. The endoscope as claimed in claim 18, wherein said endoscope is activated by moving the slidable switch in a linear direction from a first position to a second position after the connection portion is placed in the control unit.

29. The endoscope as claimed in claim 18, wherein said endoscope is activated by moving the slidable switch in a linear direction from an unlocked position to a locked position after the connection portion is placed in the control unit.

30. A system for connecting a proximal connection portion of an endoscope to a control unit of an endoscope, the connection portion comprising a male fitting member which is adapted to fit into a female fitting member placed in an opening on a surface of the main control unit, wherein said female fitting member is coupled to a crankshaft that is adapted to be operated by activating an electrical motor.

31. The system as claimed in claim 30, wherein a movement of said crankshaft causes the female fitting member to rotate and lock or unlock with said male fitting member.

32. The system as claimed in claim 30, wherein said electrical motor is manually operated through a switch provided on the control unit.

33. The system as claimed in claim 30, further comprising a sensor device which detects that the male fitting member is connected to the female fitting member and, based on said detection, activates said electrical motor to lock the male fitting member to the female fitting member.

34. The system as claimed in claim 30, wherein said crankshaft and said electrical motor are located in the control unit.

35. The system as claimed in claim 30, wherein said male fitting member is a bayonet connector.

36. The system as claimed in claim 30, wherein said female fitting member is a bayonet connector.

37. The system as claimed in claim 30, wherein said female fitting member is placed in an opening of the control unit, and wherein said opening is adapted to receive a utility cable.

38. The system as claimed in claim 30, wherein said male fitting member comprises an electrical connection portion.

39. The system as claimed in claim 30, wherein said crankshaft is a forged structure permanently affixed to the female fitting member and/or said electrical motor.

40. The system as claimed in claim 30, wherein said crankshaft is attached to said female fitting member and/or said electrical motor using screws.
